(19) United States
(12) Patent Application Publication
(10) Pub. No.: US 2021/0085096 A1
(43) Pub. Date: Mar. 25, 2021

(54) INFANT CARE APPARATUS

(71) Applicant: Thorley Industries, LLC (dba 4moms), Pittsburgh, PA (US)

(72) Inventor: Richard Juchniewicz, Pittsburgh, PA (US)

(21) Appl. No.: 17/025,582

(22) Filed: Sep. 18, 2020

Related U.S. Application Data
(60) Provisional application No. 62/902,770, filed on Sep. 19, 2019.

(51) Int. Cl. A47D 9/02; A61B 5/00; A61B 5/11; G06N 3/02

(52) U.S. Cl. CPC A47D 9/02; A61B 5/6891; A61B 5/6801; A61B 2503/04; A61B 5/742; A61B 5/11; G06N 3/02; A61B 5/7405

(57) ABSTRACT

An infant care apparatus includes an infant holder, a drive section, a biometric sensor, and a controller. The drive section is coupled to the infant holder and has a motor configured to generate one or more of an action and a motion of the infant holder. The biometric sensor is configured to observe at least one characteristic of an infant within the infant holder. The controller is configured to employ a neural network or state machine that is communicably coupled to the biometric sensor and the drive section, where the controller registers sensor data from the biometric sensor and effects, through the neural network or the state machine, a change in the one or more of the action and the motion of the infant holder.

### CROSS-REFERENCE TO RELATED APPLICATIONS

This application is a non-provisional of and claims the benefit of U.S. provisional patent application number 62/902,770 filed on Sep. 19, 2019, the disclosure of which is incorporated herein by reference in its entirety.

### BACKGROUND

### 1. Field

The disclosed embodiment relates generally to an infant care apparatus and, more particularly, to an infant care apparatus having an occupant area that is movable by a drive mechanism.

### 2. Description of Related Art

Baby swings, bouncy seats, cradles, and bassinets have been used to hold, comfort, and entertain infants and babies for many years. Prior art bouncy seats are normally constructed with a wire frame that contains some resistance to deformation that is less than or equal to the weight of the child in the seat. Thus, when the child is placed in the seat, his or her weight causes a slight and temporary deformation in the wire structure that is then counteracted by the wire frame's resistance to deformation. The end result is that the child moves up and down slightly relative to the floor. This motion can be imparted to the seat by a caregiver for the purpose of entertaining or soothing the child.

Baby swings normally function in much the same way as swing sets for older children; however, the baby swing usually has an automated power-assist mechanism that gives the swing a “push” to continue the swinging motion in much the same way a parent will push an older child on a swing set to keep them swinging at a certain height from the ground.

There are some products that have recently entered the market that defy easy inclusion into either the bouncy or swing category. One such product includes a motorized motion that can move the infant laterally, but only has a single degree of motorized freedom and is thus limited in the motion profiles that can be generated. While the seat can be rotated so that the baby is moved back and forth in a different orientation, there remains only one possible motion profile.

As a baby or infant occupies the baby swing, bouncy seat, cradle, and bassinet, the mood of the baby or infant may change (e.g., from a calm mood to crying, etc.). In other aspects, a state of the babies or infants may change (e.g., from sleeping to awake or vice versa, etc.). Generally, changes in the baby's or infant's mood or state are monitored directly by a caretaker or through a baby monitoring device which typically includes a camera and microphone that merely transmits a video and audio of the baby or infant to a remotely located monitoring device accessible to the caretaker. As the baby's mood or state changes the caretaker must physically tend to the baby or infant so as to sooth the baby or infant.

A need exists for a motorized infant support that is capable of simultaneous or independent movement in at least two directions, and can reproduce a large number of motion profiles with those two directions. A need also exists for a reactive infant support that is capable of adjusting action/motion characteristics of the infant support based on the baby's or infant's mood or state.

### BRIEF DESCRIPTION OF THE DRAWINGS

FIG. 1 is a perspective view of an infant care apparatus in accordance with aspects of the disclosed embodiment;

FIG. 1A is a side view of a portion of the infant care apparatus of FIG. 1 in accordance with aspects of the disclosed embodiment;

FIG. 2 is a perspective view of an infant care apparatus in accordance with aspects of the disclosed embodiment;

FIG. 2A is a side view of the infant care apparatus of FIG. 2 in accordance with aspects of the disclosed embodiment;

FIG. 3A is an exemplary control loop of the infant care apparatus of FIGS. 1 and 2 in accordance with aspects of the disclosed embodiment;

FIG. 3B is a schematic block diagram of a portion of the infant care apparatus of FIGS. 1 and 2 in accordance with aspects of the disclosed embodiment;

FIG. 3C is a schematic illustration of an exemplary state machine in accordance with aspects of the disclosed embodiment;

FIG. 3D is a schematic illustration of an exemplary neural network in accordance with aspects of the disclosed embodiment;

FIG. 4 is a perspective view of a portion of the infant care apparatus of FIG. 1 and/or FIG. 2 in accordance with aspects of the disclosed embodiment;

FIG. 5 is a perspective view of a portion of the infant care apparatus of FIG. 1 and/or FIG. 2 in accordance with aspects of the disclosed embodiment;

FIGS. 6A-6F are cross-sectional views of a portion of the infant care apparatus of FIG. 1 and/or FIG. 2 in accordance with aspects of the disclosed embodiment;

FIG. 7 is a perspective view of a portion of the infant care apparatus of FIG. 1 and/or FIG. 2 in accordance with aspects of the disclosed embodiment;

FIGS. 8A and 8B are perspective views of a portion of the infant care apparatus of FIG. 1 and/or FIG. 2 in accordance with aspects of the disclosed embodiment;

FIG. 9A is a side view of a portion of the infant care apparatus of FIG. 1 and/or FIG. 2 in accordance with aspects of the disclosed embodiment;

FIG. 9B is a front perspective view of a portion of the infant care apparatus of FIG. 1 and/or FIG. 2 in accordance with aspects of the disclosed embodiment;

FIG. 9C is a perspective view of a portion of the infant care apparatus of FIG. 1 and/or FIG. 2 in accordance with aspects of the disclosed embodiment;

FIG. 10A is a bottom perspective view of a portion of the infant care apparatus of FIG. 1 and/or FIG. 2 in accordance with aspects of the disclosed embodiment;

FIG. 10B is a side view of a portion of the infant care apparatus of FIG. 1 and/or FIG. 2 in accordance with aspects of the disclosed embodiment;

FIG. 10C is a bottom perspective view of a portion of the infant care apparatus of FIG. 1 and/or FIG. 2 in accordance with aspects of the disclosed embodiment;

FIG. 11 is a perspective view of a portion of the infant care apparatus of FIG. 1 and/or FIG. 2 in accordance with aspects of the disclosed embodiment;

FIG. 12 is a perspective view of the portion of the infant care apparatus of FIG. 12 in accordance with aspects of the disclosed embodiment;

FIG. 13 is a cross-sectional view of the portion of the infant care apparatus of FIG. 12 in accordance with aspects of the disclosed embodiment;

FIG. 13A is a front view of a portion of the portion of the infant care apparatus of FIG. 12 in accordance with aspects of the disclosed embodiment;

FIG. 14 is a perspective view of a portion of the infant care apparatus of FIG. 1 and/or FIG. 2 in accordance with aspects of the disclosed embodiment;

FIG. 15 is a perspective view of a portion of the portion of the infant care apparatus of FIG. 14 in accordance with aspects of the disclosed embodiment;

FIG. 16 is a perspective view of the portion of the infant care apparatus of FIG. 15 in accordance with aspects of the disclosed embodiment;

FIG. 17 is a top view of the portion of the infant care apparatus of FIG. 15 in accordance with aspects of the disclosed embodiment;

FIG. 18 is a front view of the portion of the infant care apparatus of FIG. 15 in accordance with aspects of the disclosed embodiment;

FIG. 19 is a side view of the portion of the infant care apparatus of FIG. 15 in accordance with aspects of the disclosed embodiment;

FIG. 20 is a partial perspective view of the portion of the infant care apparatus of FIG. 14 in accordance with aspects of the disclosed embodiment;

FIG. 21 is a partial perspective view of the portion of the infant care apparatus of FIG. 14 in accordance with aspects of the disclosed embodiment;

FIG. 22 is a partial perspective view of the portion of the infant care apparatus of FIG. 14 in accordance with aspects of the disclosed embodiment;

FIGS. 23A-23E are illustrative diagrams of representative motion profiles in accordance with aspects of the disclosed embodiment;

FIG. 24 is a block diagram of an exemplary control system of the infant care apparatus of FIG. 1 and/or FIG. 2 in accordance with aspects of the disclosed embodiment;

FIG. 25 is a method for imparting motion on the infant care apparatus of FIG. 1 and/or FIG. 2 in accordance with aspects of the disclosed embodiment; and

FIG. 26 is a method for effecting a change in one or more of an action and motion of infant care apparatus of FIG. 1 and/or FIG. 2 in accordance with aspects of the disclosed embodiment.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the aspects of the disclosed embodiment as it is oriented in the drawing figures. However, it is to be understood that the aspects of the disclosed embodiment may assume alternative variations and step sequences, except where expressly specified to the contrary. It is also to be understood that the specific devices and processes illustrated in the attached drawings, and described in the following specification, are simply exemplary of the aspects of the disclosed embodiment. Hence, specific dimensions and other physical characteristics related to the aspects of the disclosed embodiment are not to be considered as limiting.

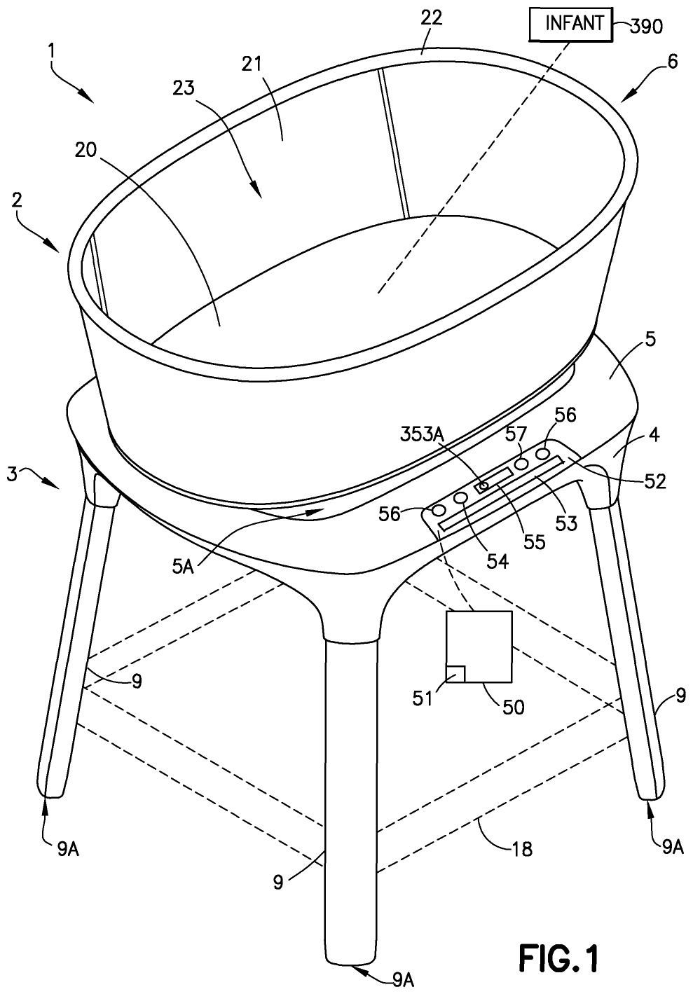

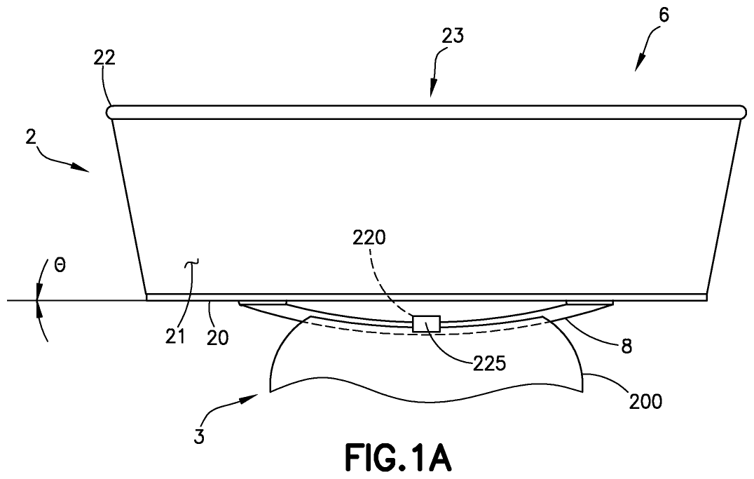

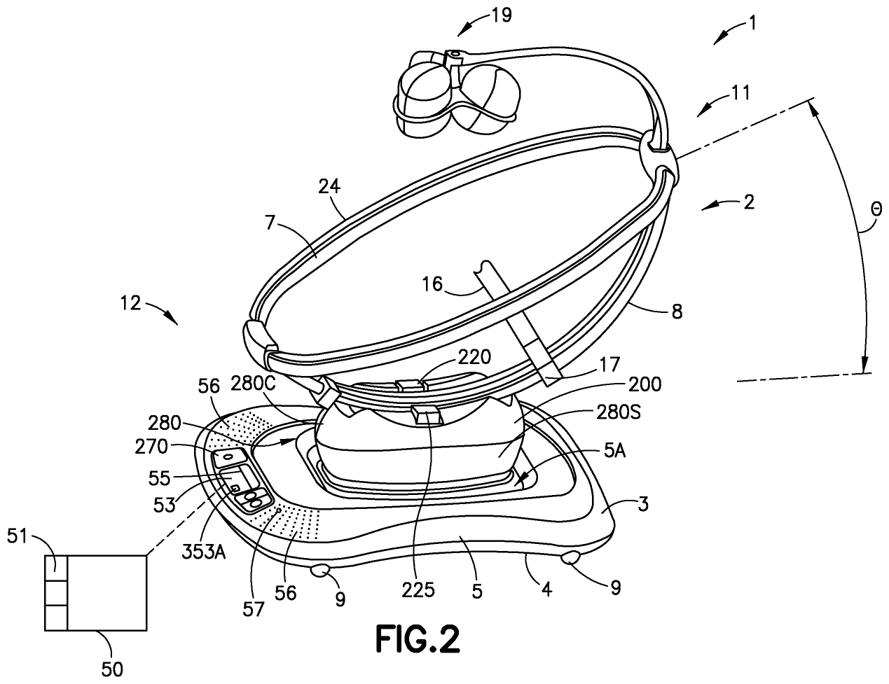

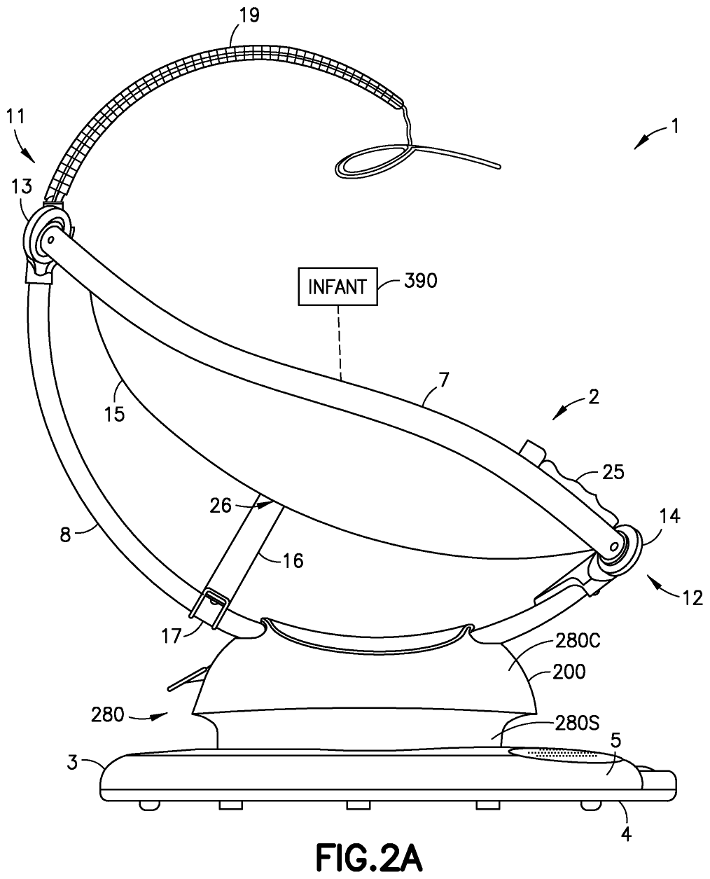

Referring to FIGS. 1, 1A, 2, and 2A an infant care apparatus 1 in accordance with aspects of the disclosed embodiment is illustrated. Although the aspects of the disclosed embodiment will be described with reference to the drawings, it should be understood that the aspects of the disclosed embodiment can be embodied in many forms. In addition, any suitable size, shape, or type of element or material could be used.

In accordance with aspects of the disclosed embodiment, the infant care apparatus 1 generally includes a base 3, an infant support 2, and an infant support coupling 200 arranged so as to releasably couple the infant support 2 to the base 3. The infant support 2 includes a mating support member 8 which is configured to be engaged with the infant support coupling 200 as will be described in greater detail below.

In one aspect, the infant support 2 may be an infant bed 6, such as a bassinet or cradle (as illustrated in FIG. 1). In other aspects, the infant support 2 may be any suitable support such as a seat (see FIG. 2). The infant bed 6 includes a bottom panel 20 and a continuous side wall 21 having a top edge 22. In one aspect, the infant bed 6 may include the mating support member 8 coupled to a bottom surface of the bottom panel 20; while in other aspects, the bottom panel may be coupled to the base substantially directly (as described herein) or in any other suitable manner. The continuous side wall 21 extends about a periphery of bottom panel 20 and is joined to the bottom panel 20 so as to define an enclosed space 23 for an infant or baby to occupy. The side wall 21 may be constructed of any suitable material such as solid fabrics/cloths, mesh fabrics, etc. While the infant bed 6 is illustrated as being elliptical in shape, the infant bed 6 may be any other suitable shape, such as, square, rectangular, circular, etc.

In another aspect, as illustrated in FIGS. 2 and 2A, the infant support 2 may be an infant seat 7 as noted above. A suitable example of the infant seat can be found in U.S. Pat. No. 10,231,555 issued on Mar. 19, 2019, the disclosure of which is incorporated herein by reference in its entirety. Although the infant seat 7 is illustrated as being elliptical in shape, the infant seat 7 may be any other suitable shape, such as, square, rectangular, circular, etc.

In some aspects, the infant bed 6 and the infant seat 7 each include the mating support member 8 which is configured to support at least the weight of an infant or baby. In some aspects, the infant bed 6 and infant seat 7 include any suitable mobile 19 (shown coupled to the infant seat 7 but may be coupled to infant bed 6 in a substantially similar manner) that may be fixed or releasably coupled to the top edge 22 of the infant bed 6, the upper end of the infant seat 7, or any other suitable location of the infant bed 6 and/or infant seat 7 in any suitable manner.

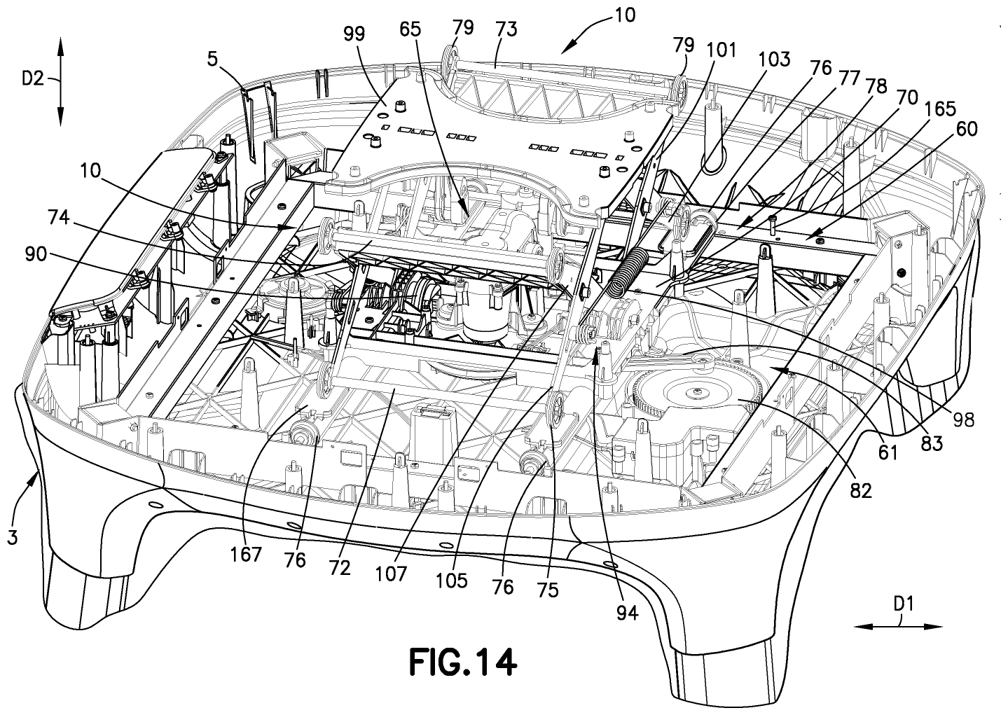

Referring to FIGS. 1, 1A, 2, and 2A, the base 3 of the infant care apparatus 1 includes a bottom support housing 4, a top enclosure 5 positioned over and at least partially covering the bottom support housing 4 a housing 280 including a cover 280C and a skirt 280S, and a housing base 281. In one aspect, the housing 280 is configured to house the infant support coupling 200. The infant support coupling 200 is disposed in the housing such that the housing cover 280C at least partially encloses the infant support coupling 200 and the skirt 280S extends from the housing cover 280C so as to circumscribe or surround at least a portion of the movable stage 10 that extends through a surface 5A of the top enclosure 5. The housing base 281 is configured to couple the infant support coupling 200 to a movable stage 10 (FIG. 14) as will be further described herein. The top enclosure 5 includes the surface 5A which at least partially covers an opening through which the movable stage 10, supported on the bottom support housing 4, extends as will be further described herein. The surface 5A may be an articulated surface configured so that the opening formed therein moves with the movable stage 10.

In one aspect, the base 3 may have fixed or detachable legs 9. In one aspect, the legs 9 may be adjustable to raise or lower a height of the infant care apparatus 1 relative to, e.g., a floor surface or table on which the infant care apparatus 1 is placed. The legs 9 include feet 9A that are contoured or otherwise shaped and sized so that the legs 9 slide easily across a floor surface. For example, the feet 9A may have curved edges to substantially avoid snagging of the feet 9A on the flooring surface as the infant care apparatus 1 slides across the floor surface under the influence of an external motive force. In one aspect, the base 3 may further include a storage basket 18 provided to storage infant or baby gear, accessories, etc. The storage basket 18 may be mounted to the legs 9 or any other suitable portion of the infant care apparatus 1. In one aspect, the base 3 may include a portable music player dock 55, with speakers 56 and an input jack 57, for playing music or other pre-recorded sounds.

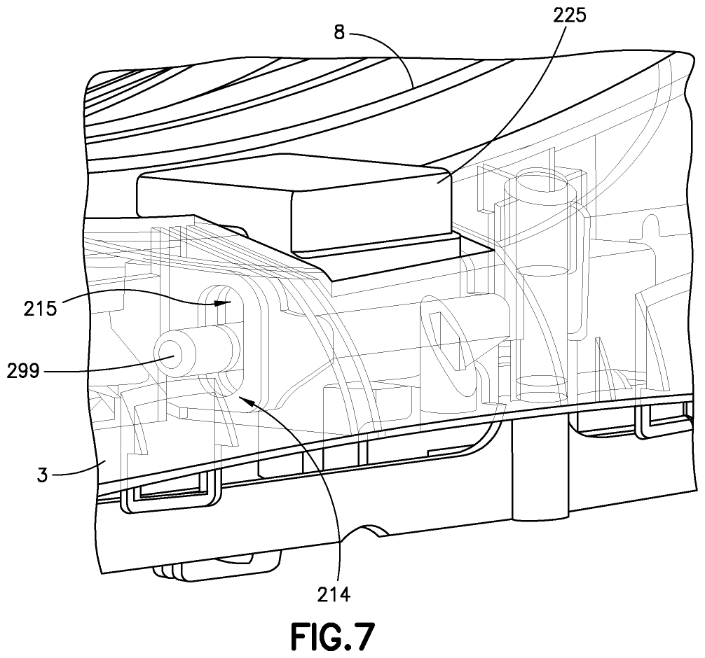

Referring now to FIGS. 1A, 2, 4, 5, 6A-6F, and 7 the mating support member 8 of the infant support 2 is configured so as to be releasably coupled to the base 3. Coupling of the infant support 2 is described herein with respect to the infant bed 6, however, it should be understood that in some aspects, the infant seat 7 may be coupled to the base 3 in a substantially similar manner using the mating support member 8 shown in FIGS. 2 and 2A. As noted above, the infant care apparatus 1 includes the infant support coupling 200 arranged so as to releasably couple the mating support member 8 of the infant support 2 to the base 3. The infant support coupling 200 includes a movable support 210 and automatically actuable grip members 220, 225 such as on placement of the infant bed 6 onto the infant support coupling 200. In other aspects, the infant support coupling 200 may have any suitable configuration such as that described in U.S. patent application Ser. No. _______ , filed on _______ having attorney docket number 1252P015822-US (PAR) and being titled “Infant Care Apparatus”, the disclosure of which is incorporated herein by reference in its entirety.

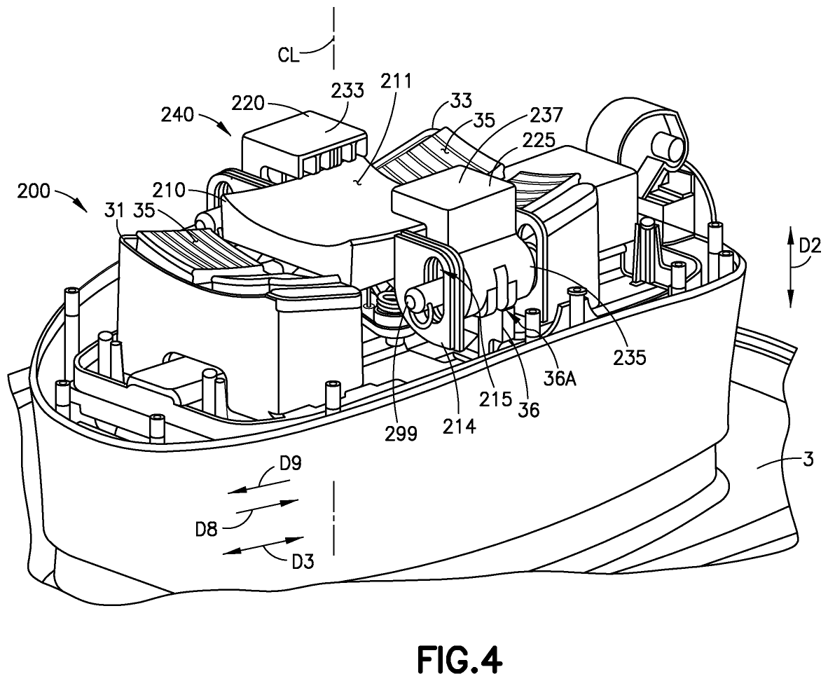

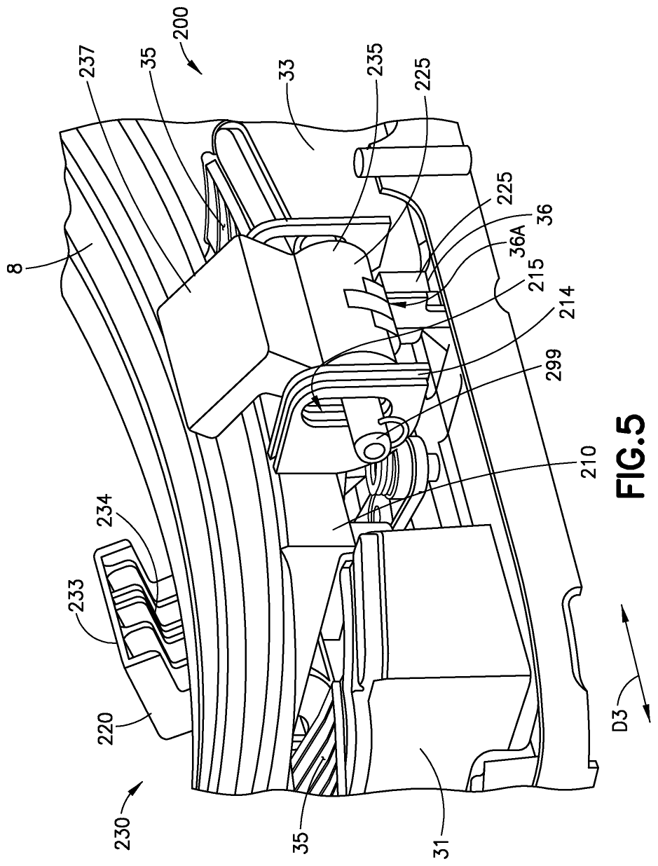

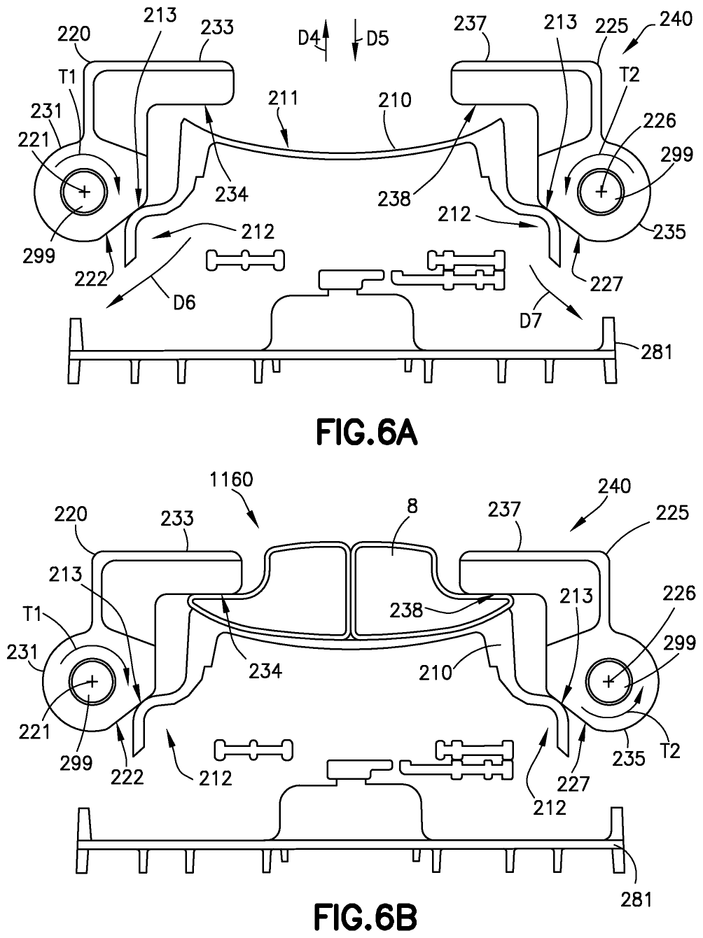

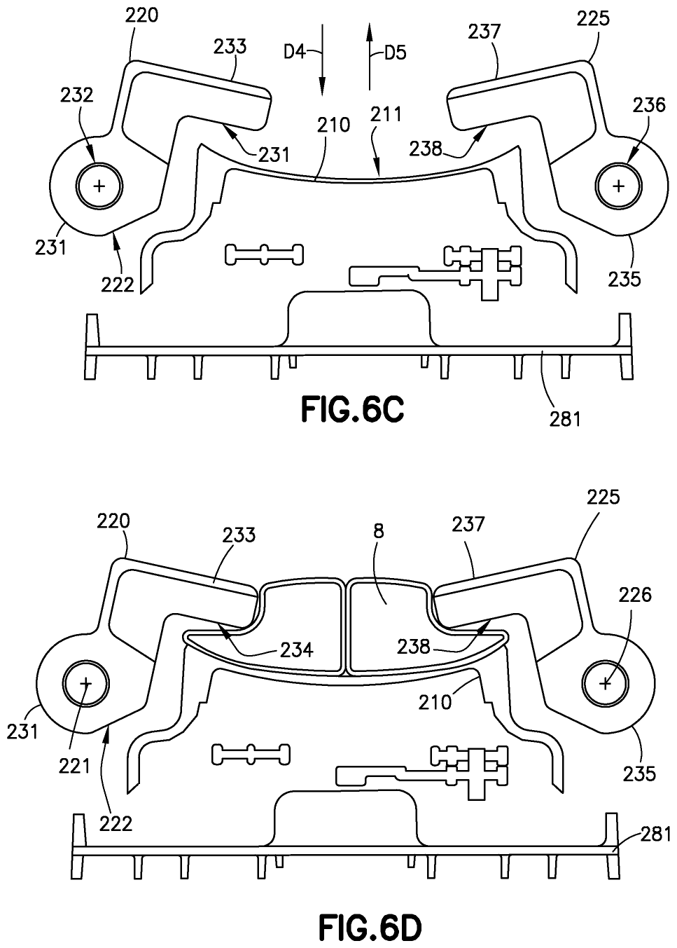

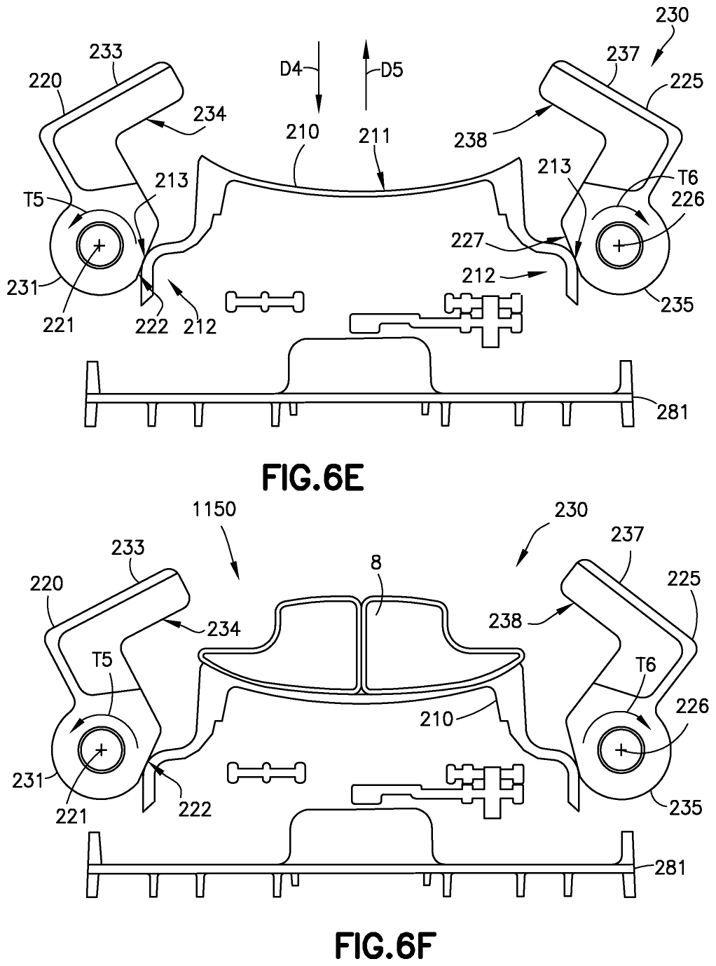

With particular reference to FIGS. 4 and 5, the movable support 210 is movably connected to the base 3 in any suitable manner so as to move in direction D2. The movable support 210 is disposed so as to form a support seat 211 that engages and supports the mating support member 8 of the infant support 2. The movable support 210 includes ribs 214 which couple to the base 3. The ribs 214 include a slotted hole 215 through which a pin 299 is inserted to constrain motion of the movable support 210 in direction D2. The slotted hole 215 has an elongated shape so that the movable support 210 may move between a first raised position 1150 (FIG. 6F) and a second lowered position 1160 (FIG. 6B) in direction D2 as will be described in greater detail below. The movable support 210 further includes a camming mechanism 212 (see, at least FIG. 6A) having camming surfaces 213 which are configured to interface with the automatically actuable grip members 220, 225 so as to automatically actuate the automatically actuable grip members 220, 225 between a clamped or closed position 240 (FIG. 6A) and an unclamped or open position 230 (FIG. 6F).

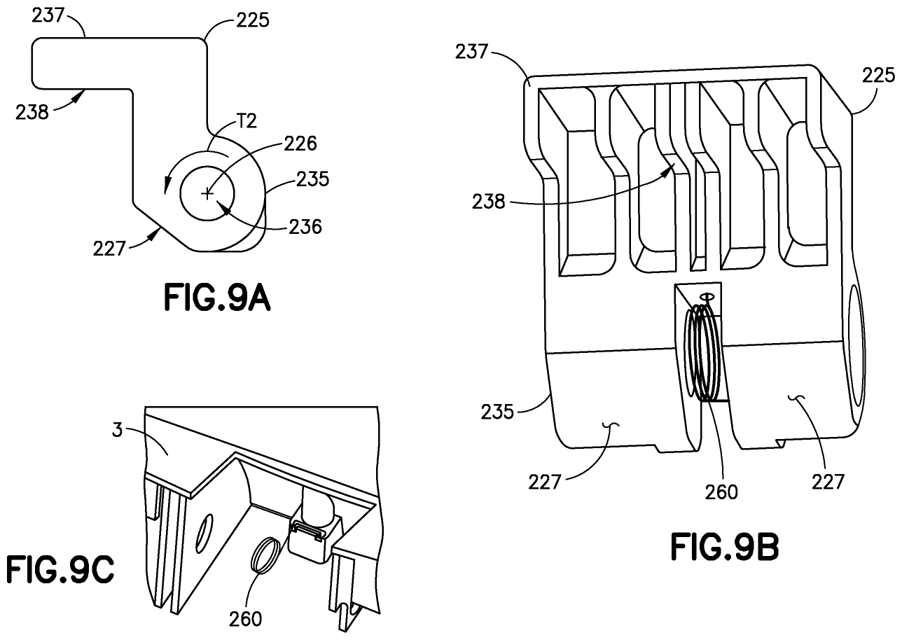

Referring to FIGS. 1A, 2, 4, 5, 6A-6F, 7, 8A-8B, and 9A-9C, the automatically actuable grip members 220, 225 each include a base 231, 235 with an aperture 232, 236, through which a respective pin 299 extends, and cam follow surfaces 222, 227. Clamp arms 233, 237 extend from the base 231, 235 and include gripping surfaces 234, 238. The automatically actuable grip members 220, 225 are coupled to a respective pin 299 so as to rotate relative to both the movable support 210, and the base 3 between the open position 230 and the closed position 240 (as seen best in FIGS. 6A-6F). In one aspect, the automatically actuable grip members 220, 225 are coupled to their respective pin 299 so as to freely rotate relative to the pin 299; while in other aspects, the automatically actuable grip members 220, 225 and the respective pin 299 may rotate as a unit relative to the slotted hole 215 and the movable support 210. The automatically actuable grip members 220, 225 are disposed with respect to the infant support 2 to effect gripping of the infant support 2 with gripping surfaces 234, 238 (FIG. 9B) when the infant support 2 is positioned on the support seat 211. The automatically actuable grip members 220, 225 actuating between the open position 230 and the closed position 240 captures and releases the mating support member 8 of the infant support 2. The automatically actuable grip members 220, 225 are automatically actuable between the open and closed positions 230, 240, by action of the movable support 210.

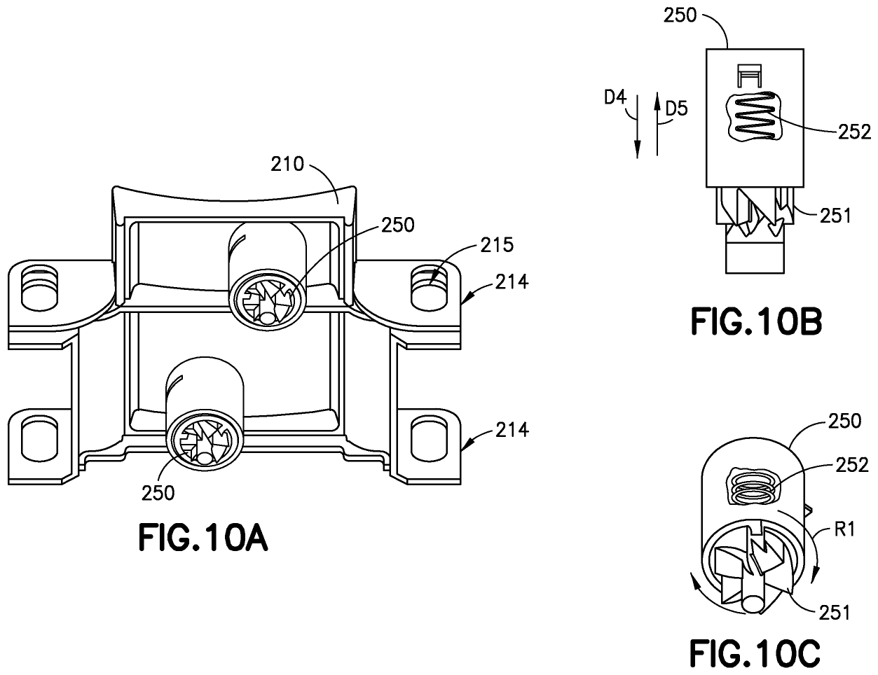

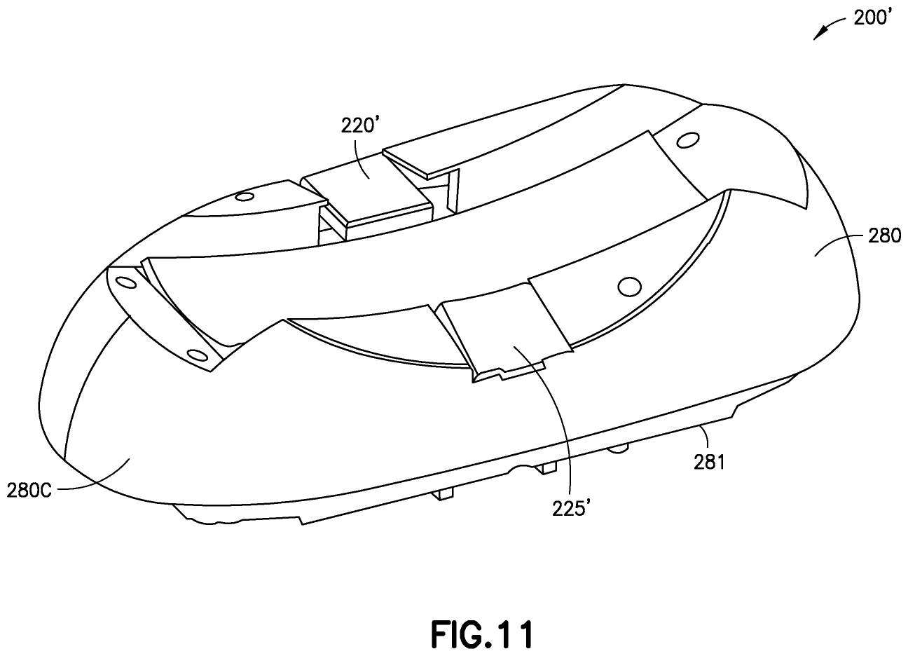

For example, referring also to FIGS. 10A-10C, the infant care apparatus 1 may further include at least one toggle mechanism 250. In one aspect, the at least one toggle mechanism 250 may form an indicator to indicate the position of the movable support 210. For example, the at least one toggle mechanism 250 may emit an aural or tactile signal to indicate the position. In one aspect, the movable support 210 may be supported on at least one toggle mechanism 250 which is configured to toggle the movable support 210 between the first raised position 1150 and the second lowered position 1160. The at least one toggle mechanism 250 utilizes an angled tooth cam 251 and a spring 252 to toggle between first raised position 1150 and the second lowered position 1160. For example, when the movable support 210 is lowered in direction D4 (FIGS. 6A-6F and 10B) (such as when the infant support 2 is being coupled to the base 3), the at least one toggle mechanism 250 is compressed and the angled tooth cam 251 rotated in direction R1. In this position, the spring 252 within the at least one toggle mechanism 250 is loaded with the angled tooth cam 251 in a compressed and locked position. In this position both the at least one toggle mechanism 250 and the movable support 210 supported thereon are in the lowered state. When the movable support 210 is moved in direction D5 (FIGS. 6A-6F and 10B) again (such as when removing the infant support 2), the at least one toggle mechanism 250 is compressed which rotates the angled tooth cam 251 in direction R1 unlocking the at least one toggle mechanism 250 and allowing the spring 252 of the at least one toggle mechanism 250 to move the movable support 210 in direction D5 (FIGS. 6A-6F and 10B).

With the at least one toggle mechanism 250 (and thus the movable support 210) in the raised position 1150, the automatically actuable grip members 220, 225 are in and remain in the open position 230 through interaction between the camming mechanism 212 and the cam follower surfaces 222, 227 of the automatically actuable grip members 220, 225. With the automatically actuable grip members 220, 225 in the open position 230, the mating support member 8 of the infant support 2 is free to be removed or placed within the support seat 211 of the movable support 210 so as to mount the infant support 2 to the base 3. In order to bias the automatically actuable grip members 220, 225 in the open position 230, the cam follow surfaces 222, 227 of the automatically actuable grip members 220, 225 are configured to interface with the camming surfaces 213 of the camming mechanism 212. For example, without the infant support 2 present on the support seat 211, the movable support 210 is in the first raised position 1150 such that the camming surfaces 213 of the camming mechanism 212 are engaged with and biasing the cam follower surfaces 222, 227 of the automatically actuable grip members 220, 225 in direction T5 and direction T6, respectively, to the open position 230 against the biasing force of torsion springs 260. As the mating support member 8 of the infant support 2 is placed on the movable support 210 by a user and the movable support 210 is moved in direction D4 into the second lowered position 1160, the camming surfaces 213 of the camming mechanism 212 are disengaged from the cam follow surfaces 222, 227 (i.e., lowered such that the cam follow surfaces 222, 227 of the automatically actuable grip members 220, 225 follow or slide along the camming surfaces 213 of the camming mechanism 212 in respective direction D6 and direction D7). The torsion springs 260 of the respective automatically actuable grip members 220, 225 effects rotation of the respective automatically actuable grip members 220, 225 in respective direction T1 and direction T2. The respective torsion springs 260 biases the automatically actuable grip member 220 in direction T1 and the automatically actuable grip member 225 in direction T2 about respective pivot axes 221, 226 to place the automatically actuable grip members 220, 225 in the closed position 240.

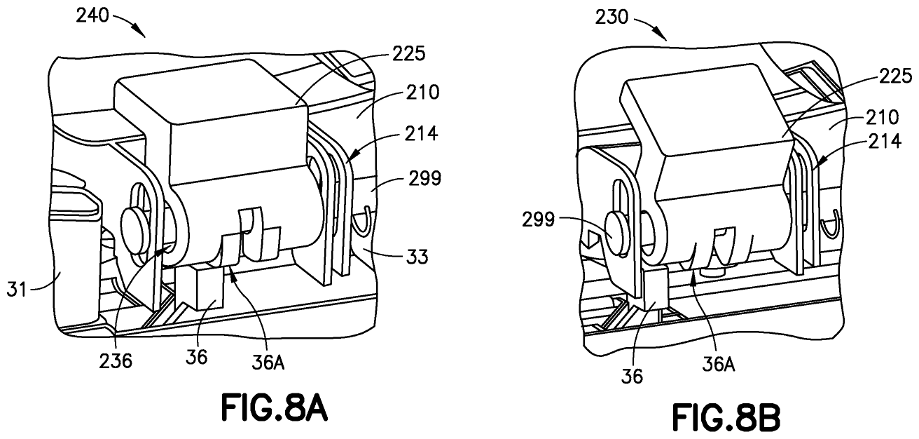

Referring to FIGS. 4, 5, and 8A-8B in one aspect, the infant support coupling 200 includes a first recline locker 31 and a second recline locker 33 each including locking pads 35 which are configured to engage the mating support member 8 so as to lock a position of the mating support member 8 relative to the base 3 and setting the angle θ (FIGS. 1A and 2). As may be realized, the angle θ is illustrated as substantially zero in FIG. 1A but may be increased or decreased so that the infant bed is substantially level (e.g., the infant support surface of the infant bed 6 is substantially in a plane parallel to the plan of the horizon) so as to compensate for any inclination of the surface upon which the infant care apparatus 1 is placed. The first recline locker 31 and second recline locker 33 are substantially similar to the locking mechanism described in U.S. Pat. No. 10,231,555 previously incorporated herein by reference. The locking pads 35 may be manufactured from rubber or any other suitable material. The first recline locker 31 and the second recline locker 33 are configured to removably engage the locking pads 35 with the mating support member 8 positioned within the support seat 211 by movement of a Z-linkage (not shown). Movement of the Z-linkage causes movement of both the first recline locker 31 and the second recline locker 33 in direction D12 to lock and release the mating support member 8 relative to the base 3. For example, to lock the mating support member 8 relative to the base 3, the Z-linkage drives the first recline locker 31 in direction D9 and the second recline locker 33 in direction D8 such that the first recline locker 31 and the second recline locker 33 move toward a centerline CL of the infant support coupling 200. The mating support member 8 is released when the Z-linkage is actuated to drive the first recline locker 31 in direction D8 and the second recline locker in direction D9 away from the centerline CL of the infant support coupling 200. The first recline locker 31 and the second recline locker 33 may include lock members 36 to lock the automatically actuable grip members 220, 225 in place. The lock members 36 are configured to move with the first recline locker and the second recline locker 33 in direction D3. For example, when the second recline locker 33 is moved in direction D8 to lock the mating support member 8 relative to the base 3, the lock member 36 is also moved in direction D8 and positioned under the automatically actuable grip member 225. The automatically actuable grip member 225 includes a lock surface 36A (FIG. 8B) that interfaces with the lock member 36 and “locks” the automatically actuable grip member 225 (i.e., prevents rotation of the automatically actuable grip member 225). The lock members 36 are coupled to the movement linkage of the recline lockers 31, 33 so as to move between locked and unlocked positions coincident with the recline lockers 31, 33 being engaged and disengaged.

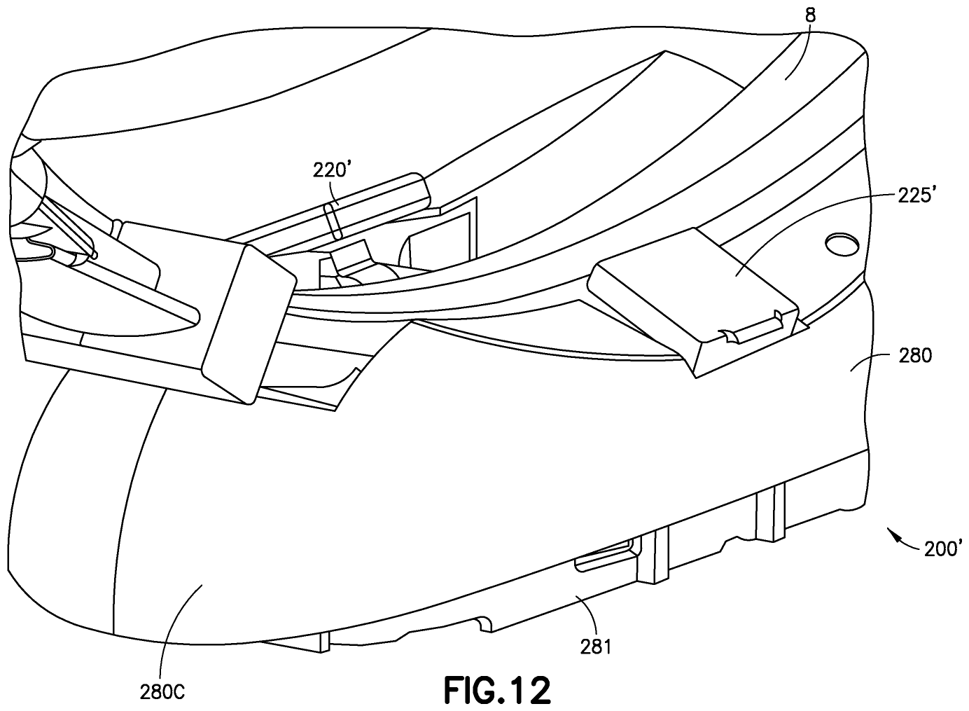

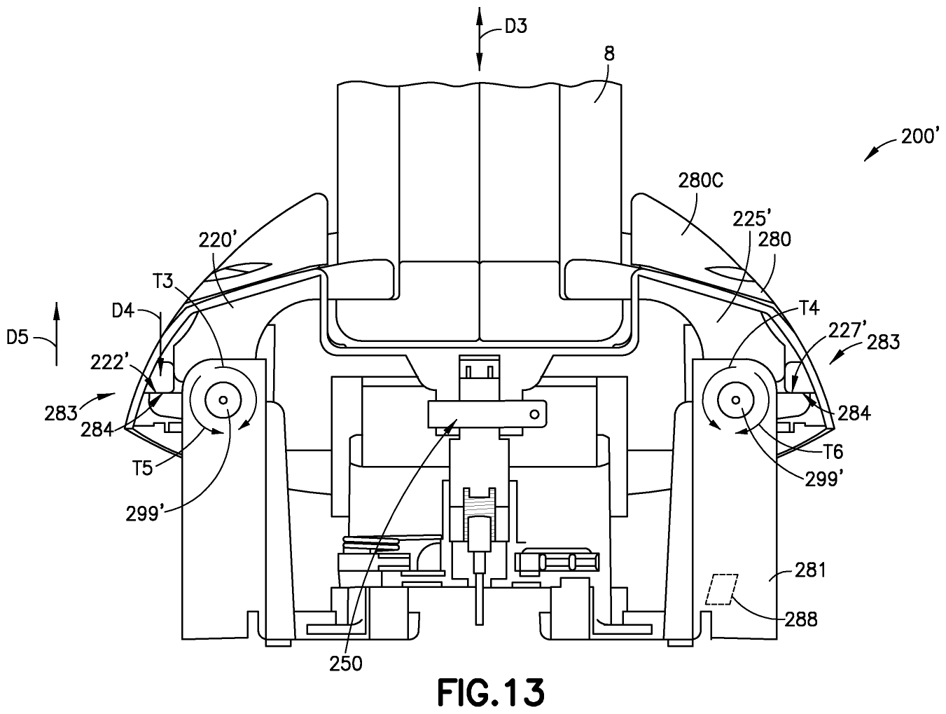

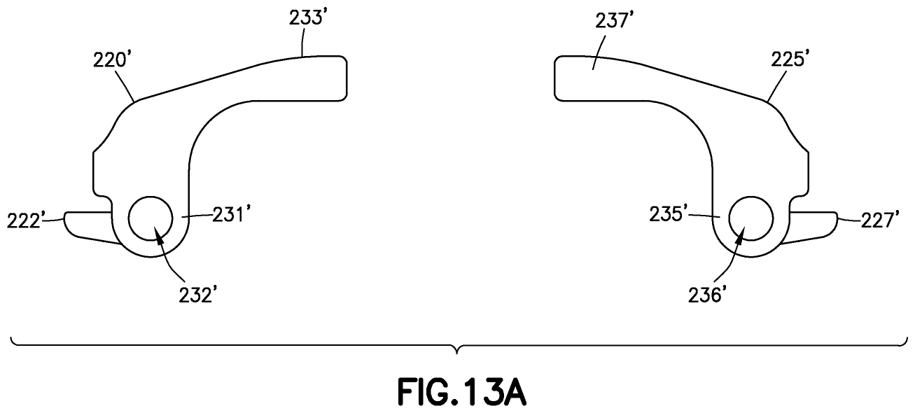

Referring now to FIGS. 11-13, infant support coupling 200′ is illustrated in accordance with another aspect of the disclosed embodiment. The infant support coupling 200′ is substantially similar to infant support coupling 200 unless where noted below. In this aspect, the infant support coupling 200′ includes automatically actuable grip members 220′, 225′, and the housing cover 280C of the housing 280 acts as the movable support 210 described above. Here, the housing cover 280C is movably coupled to the base 3 in any suitable manner, such as, by the housing base 281 such that the housing cover 280C moves in direction D2 relative to the housing base 281 fixedly mounted to the base 3. It is noted that the skirt 280S is coupled to the housing base 281 independent of the housing cover 280C so that the housing cover 280C moves in direction D2 relative to the skirt 280S. The skirt 280S extends from the housing base 281 (or with respect to the infant support coupling 200′) so as to circumscribe or surround at least a portion of the movable stage 10 that extends through the surface 5A. The housing cover 280C includes camming mechanism 283 with camming surfaces 284 to effect automatic actuation of the automatically actuable grip members 220′, 225′ as will be described below.

The automatically actuable grip members 220′, 225′ each include a base 231′, 235′ with an aperture 232′, 236′, through which a respective pin 299′ extends, and cam followers 222′, 227′ extending from the base 231′, 235′. Clamp arms 233′, 237′ extend from the base 231′, 235′ and include gripping surfaces 234′, 238′. The automatically actuable grip members 220′, 225′ are coupled to the respective pins 299′ so as to rotate relative to the housing cover 280C (and the base 3) between the open position 230 and the closed position 240. Here, the camming surfaces 284 of the camming mechanism 283 are engaged with and biasing the cam followers 222′, 227′ of the automatically actuable grip members 220′, 225′ in the open position 230 when the housing cover 280C is lowered in direction D4. As the mating support member 8 of the infant support 2 is placed on the movable support 210 by a user and the movable support 210 is lowered in direction D4 into the second position, the camming surfaces 284 of the camming mechanism 283 are lowered in direction D4 such that the cam followers 222′, 227′ of the automatically actuable grip members 220′, 225′ are rotated in respective directions T5 and direction T6 which forces the automatically actuable grip members 220′, 225′ into the open position 230. A torsion spring integrated into the automatically actuable grip members 220′, 225′ effects rotation of the automatically actuable grip members 220′, 225′ in respective direction T3 and direction T4 on the automatically actuable grip members 220′, 225′ to force them into the closed position 240 when the camming mechanism 283 is disengaged (i.e., the housing cover 280C is toggled into the raised position). The infant support coupling 200′ may further include shock towers 288 to absorb any impacts and retain stability of the infant support coupling 200′.

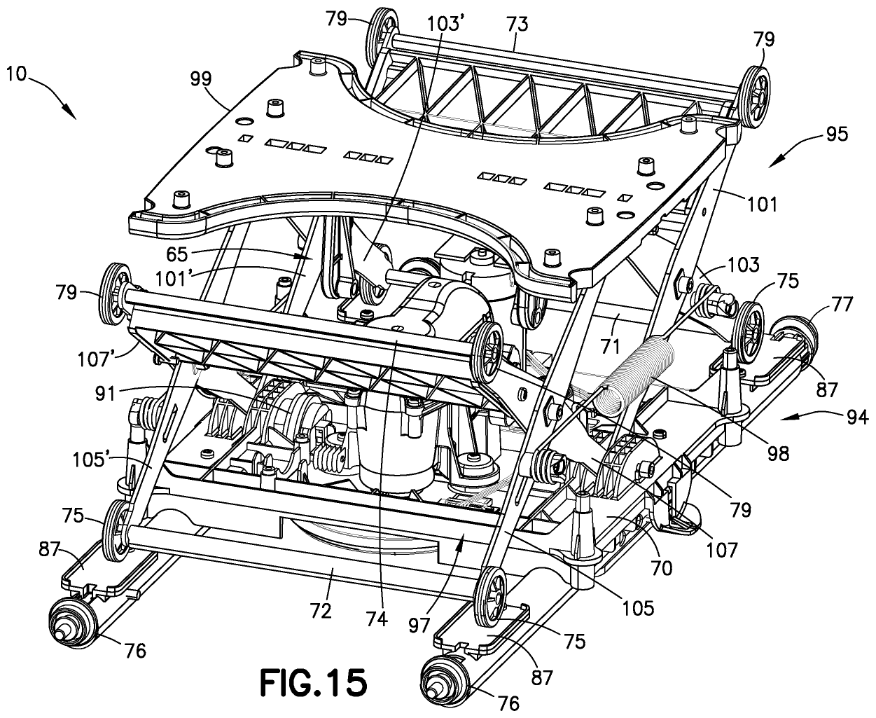

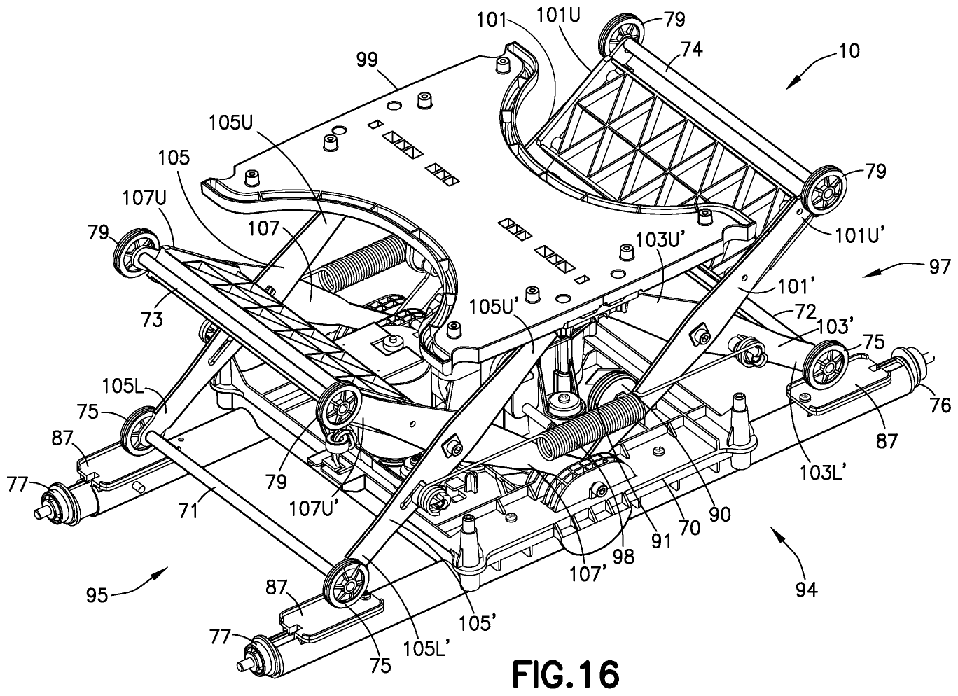

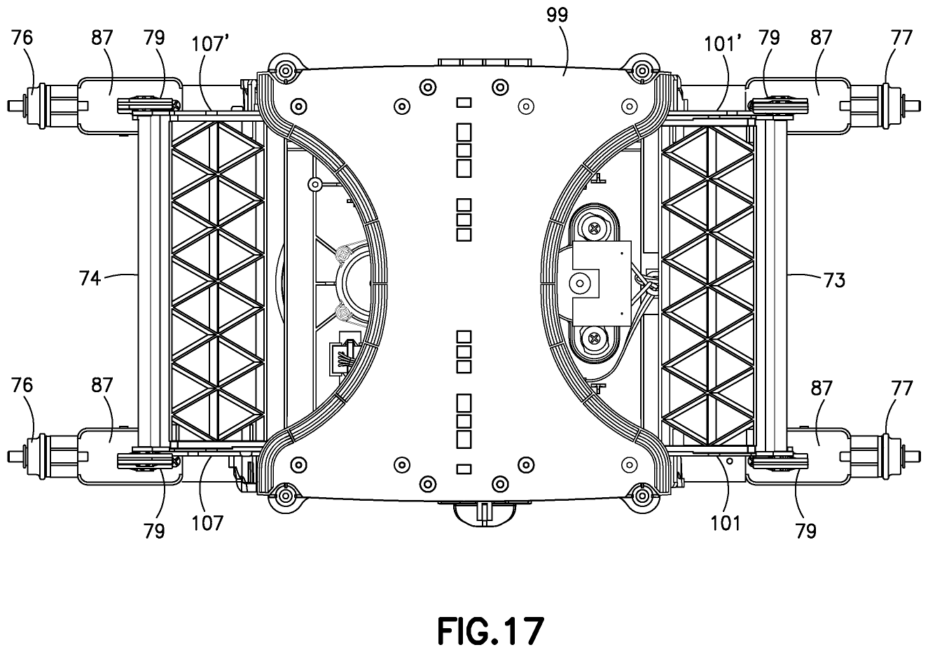

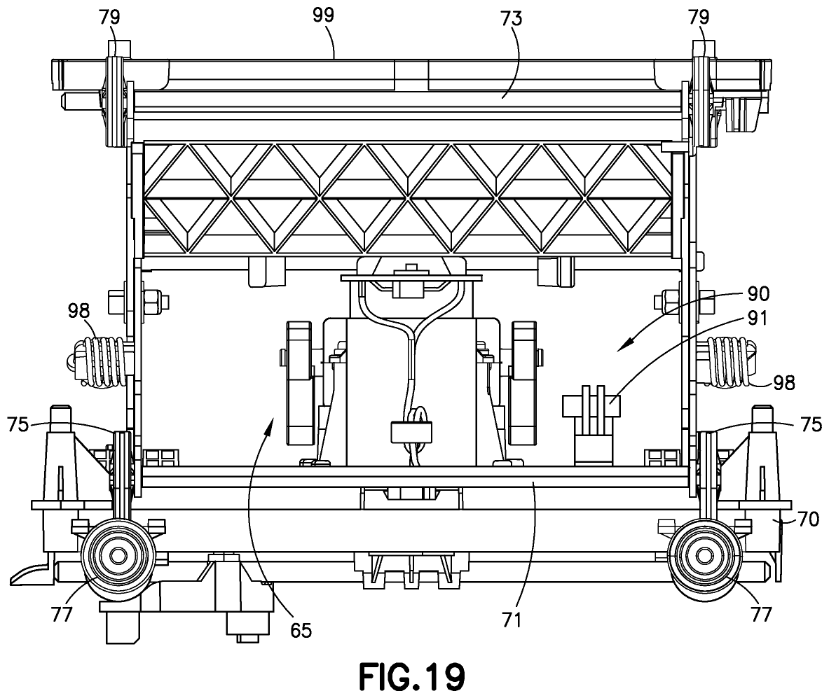

Referring now to FIGS. 14-19, in one aspect, the infant care apparatus 1 may include a drive mechanism 60 coupled to the base 3, a vibratory mechanism 90, a movable stage 10 movably mounted to the base 3, and a control system 50 (including controller 51) communicably coupled to each of the drive mechanism 60 and the vibratory mechanism 90. In one aspect, the movable stage 10 includes a first (here rigid) platform 70 and a support platform 99. A lifting motion assembly 65, here, e.g., a double scissor mechanism 94 having a first scissor mechanism 95 operatively coupled to a second scissor mechanism 97 though any other lifting motion assembly may be provided (see FIG. 15), movably joins the support platform 99 and the first platform 70. The support platform 99 is configured for coupling with the housing base 281 and/or substantially directly to the infant support coupling 200 in any suitable manner, such as, with mechanical fasteners, chemical fasteners, or a combination thereof. A suitable example of the double scissor mechanism 94 can be found in U.S. Pat. No. 10,231,555 previously incorporated herein by reference. The first platform 70 includes at least one wheel 76 suitably disposed thereon such that the first platform 70 is rollingly supported by the at least one wheel 76. Rails 78 are fixably attached to the bottom support housing 4 of the base 3. The rails 78 are configured to receive and support the at least one wheel 76 of the first platform 70 so that the movable stage 10 is configured to reciprocate along the rails 78 in a first direction D1 (such as a horizontal direction). In one aspect, the at least one wheel 76 may be a flanged wheel 77, the flange of which rides along the respective rail 78 within a corresponding groove of the rail 78 so as to linearly guide the movable stage 10 along the rails 78. In one aspect, the movable stage 10 may reciprocate along the rails 78 about three inches, while in other aspects, the movable stage 10 may reciprocate along the rails 78 any suitable distance such as more or less than about 3 inches.

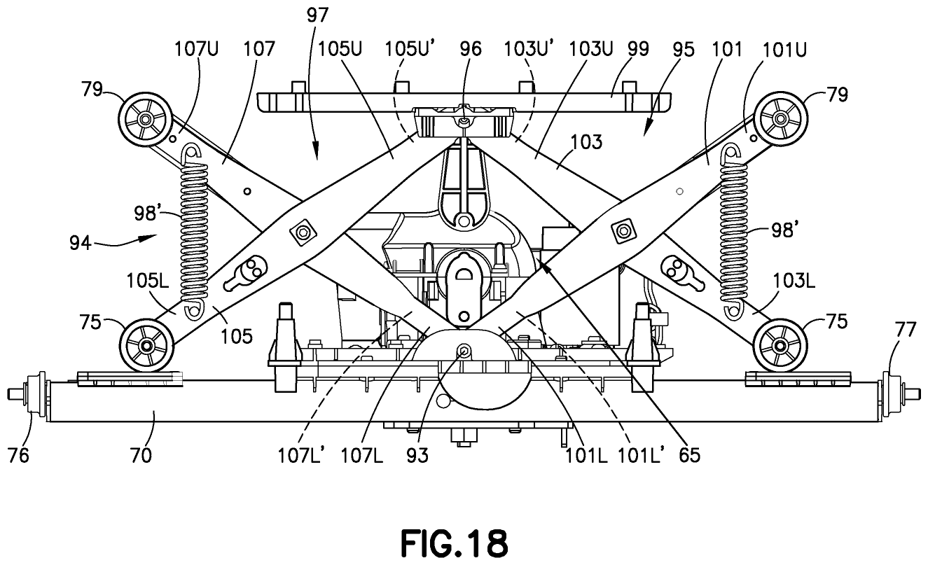

The lifting motion assembly 65 (here the first scissor mechanism 95 and the second scissor mechanism 97) is attached between the first platform 70 and the support platform 99 so as to couple the first platform 70 to the support platform 99. Here, the first scissor mechanism 95 includes a first pair of spaced-apart parallel members 101, 101′ and a second pair of spaced-apart parallel members 103, 103′. The second scissor mechanism 97 includes a third pair of spaced-apart parallel members 105, 105′ and a fourth pair of spaced-apart parallel members 107, 107′. Lower ends 101L, 101L′ of the first pair of spaced-apart parallel members 101, 101′ and lower ends 107L, 107L′ of the fourth pair of spaced-apart parallel members 107, 107′ are rotatably pinned to each other and to the first platform 70 about axis 93 (FIG. 18). Likewise, upper ends 103U, 103U′ of the second pair of spaced-apart parallel members 103, 103′, and upper ends 105U, 105U′ of the third pair of spaced-apart parallel members 105, 105′ are rotatably pinned to each other and to the support platform 99 about axis 96 (FIG. 18). The first pair of spaced-apart parallel members 101, 101′ are pivotally secured at a central portion thereof to the second pair of spaced-apart parallel members 103, 103′ via horizontal pivot pins, or the like. Correspondingly, the third pair of spaced-apart parallel members 105, 105′ are pivotally secured at a respective central portion to the fourth pair of spaced-apart parallel members 107, 107′ via horizontal pivot pins, or the like. When the support platform 99 is displaced, e.g., in a second direction D2 (such as a vertical direction), as will be described in greater detail hereinafter, the first scissor mechanism 95 and the second scissor mechanism 97 move in a crossed fashion relative to the pivot pins such that the double scissor mechanism 94 extends between the first platform 70 and the upwardly displaced support platform 99. While the lifting motion assembly 65 connected to the movable stage 10 has been illustrated and described herein as including a double scissor mechanism 94, the movable stage 10, in other aspects, may have any suitable configuration for providing a reciprocating movement in the second direction D2.

Still referring to FIGS. 14-19, in one aspect, another motion assembly 61 (lateral) is operably connected to the movable stage 10. A suitable example includes first and second horizontal bars 71, 72 are provided, where the first horizontal bar 71 extends transversely between the lower ends 103L, 103L′ of the second pair of spaced-apart parallel members 103, 103′, and the second horizontal bar 72 extends between the lower ends 105L, 105L′ of the third pair of spaced-apart parallel members 105, 105′ to provide structural stability. In addition, the first and second horizontal bars 71, 72 may further include bearing wheels 75 at their ends that interface with travel surfaces 87 of the first platform 70 of the movable stage 10 for supporting the double scissor mechanism 94 and the support platform 99. Third and fourth horizontal bars 73, 74 are provided, where the third horizontal bar 73 extends transversely between the upper ends 101U, 101U′ of the first pair of spaced-apart parallel members 101, 101′, and the fourth horizontal bar 74 extends between the upper ends 107U, 107U′ of the fourth pair of spaced-apart parallel members 107, 107′. The third and fourth horizontal bars 73, 74 may include bearing wheels 79 at their ends for engaging and supporting the infant support 2 coupled to the infant support coupling 200 (described above). In another aspect, the support platform 99 may be extended so that the bearing wheels 79 engage and support on the support platform 99 as illustrated in phantom in FIG. 18.

In one aspect, the movable stage 10 may be provided with at least one resilient element 98, such as a tension spring, fixably attached between two or more of the pair of spaced-apart parallel members 101, 101′ 103, 103′ 105, 105′ 107, 107′. The resistive mechanical element(s) 98 may be provided and configured so as to assist a lifting motion assembly 65 (described below) in extending or retracting the double scissor mechanism 94 in the second direction D2. For example, the resistive mechanical element(s) 98 may be coupled to the lower end 103L, 103L′ of second pair of spaced-apart parallel members 103, 103′ and the lower end 105L, 105L′ of the third pair of spaced-apart parallel members 105, 105′ (FIGS. 14-16. In this configuration, the resilient element 98 applies a tension force to the second pair of spaced-apart parallel members 103, 103′ and the third pair of spaced-apart parallel members 105, 105′ and pulls the relevant portions toward each other, assisting, e.g., upward vertical motion of the lifting motion assembly 65. In another example, resilient element 98′ (FIG. 18) may be a compression spring positioned so as to apply an expansion force to the double scissor mechanism 94 pushing the relevant portions apart, assisting, e.g., upward vertical motion of the lifting motion assembly 65. The positions of the resilient element 98, 98′ described above are not to be construed as limiting as the exact location of the attachment of the resilient element 98, 98′ to the double scissor mechanism 94 and can be varied with similar results. The resilient element 98, 98′ also has the benefit of counteracting or increasing the effects of gravity by acting to reduce or increase downward movement, respectively.

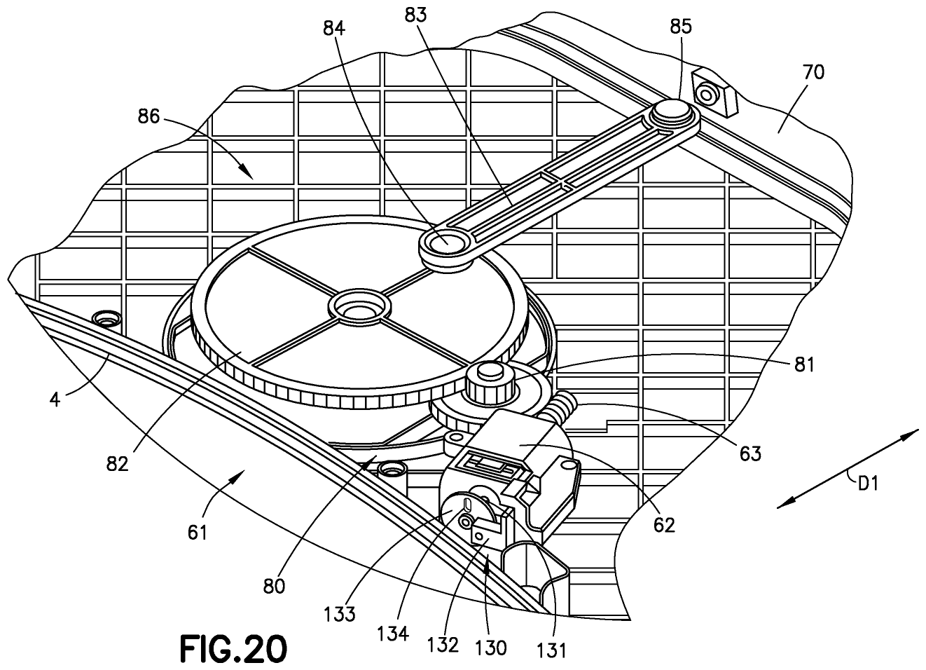

Referring to FIGS. 20-22, and with continuing reference to FIGS. 14-19, as noted above, the infant care apparatus 1 includes the drive mechanism 60 coupled to and supported by the bottom support housing 4 of the base 3. The drive mechanism 60 includes the lateral motion assembly 61 imparting a first cyclic motion in a first direction D1 to the movable stage 10 (e.g., providing lateral motion) and the lifting motion assembly 65 imparting a second cyclic motion in a second direction D2 to the movable stage 10 (e.g., providing lifting motion) as noted. As may be realized, the respective first and second cyclic motions imparted by the corresponding motion assemblies 61, 65 are directed in orthogonal directions and are thus kinematically independent relative to each other.

The lateral motion assembly 61 includes a driving portion with a first motor 62 having a drive shaft 63 and being dependent from the base 3, and a slide crank assembly 80 mounted to the bottom support housing 4 of the base 3. The first motor 62 is configured to impart the first cyclic motion in the first direction D1 to the movable stage 10. The slide crank assembly 80 includes a gearing assembly 86 having a set of first gears 81 operatively coupled to the drive shaft 63 of the first motor 62 and a second gear 82 operatively coupled to the set of first gears 81. A crank member 83, having a first end 84 and a second end 85, couples the second gear 82 to the first platform 70 to impart the first cyclic motion provided by the first motor 62 on the first platform 70 of the movable stage 10. For example, the first end 84 of the crank member 83 may be rotationally coupled to a point on the outer circumference of the second gear 82, and the second end 85 of the crank member 83 may be rotationally coupled to the first platform 70.

In operation, actuation of the first motor 62 causes rotation of the first gears 81 which in turn causes rotation of the second gear 82. The rotation of the second gear 82 drives the crank member 83 coupled to the outer circumference of the second gear 82. As the first end 84 of the crank member 83 rotates about the second gear 82, the first platform 70 is pushed and pulled by the second end 85 of the crank member 83 in the first direction Dl. This operation effects reciprocation of the driven portion of the motion assembly 61 joined to and thus imparting lateral motion to the movable stage 10 in the first direction along, e.g., the rails 78. Accordingly, the lateral motion assembly 61 is configured such that a single motor (i.e., the first motor 62) moves the first platform 70 in the first direction (e.g., horizontally) with the first motor 62 only running in a single direction, thereby eliminating backlash in the system. The system for controlling the lateral motion assembly 61 to achieve the desired motion profile will be discussed in greater detail hereinafter.

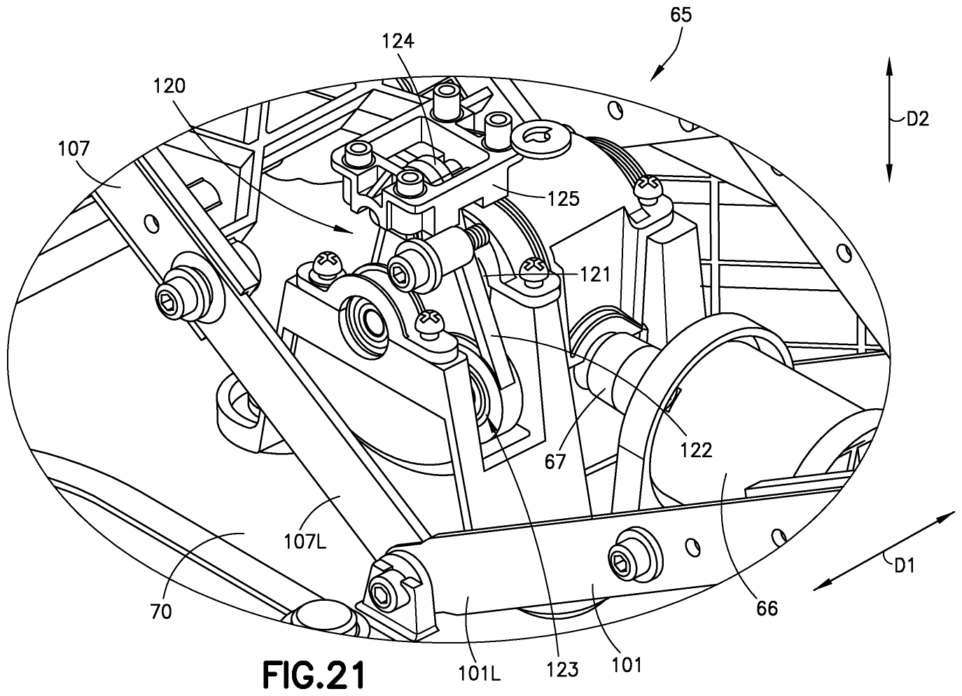

Still referring to FIGS. 14-22, the lifting motion assembly 65 is disposed on the first platform 70 of the movable stage 10 and is configured to impart the second cyclic motion to at least part of the movable stage 10 in the second direction D2 independent of the first cyclic motion in the first direction imparted by the lateral motion assembly 61. The lifting motion assembly 65 includes a second motor 66 separate and distinct from the first motor 62, disposed on the first platform 70. The second motor 66 includes a drive shaft 67 operatively coupled to a worm gear drive assembly 120. The worm gear drive assembly 120 converts rotation of the drive shaft 67 to rotational movement of an output member 121 that is perpendicular to the rotation of the drive shaft 67. A vertical yoke 122 is rotatably attached at a first end 123 thereof to the output member 121 in a manner such that the vertical yoke 122 vertically reciprocates an attachment member 125 attached to a second end 124 of the vertical yoke 122 along direction D2 shown in FIG. 21. The attachment member 125 is configured to couple to and drive/support the support platform 99 (along with the wheels 79). Accordingly, the lifting motion assembly 65 is configured such that a single motor (i.e., the second motor 66) moves the support platform 99 in the second direction D2 (e.g., vertically) with the second motor 66 only running in a single direction, thereby eliminating backlash in the system. The system for controlling the lifting motion assembly 65 to achieve the desired motion profile will be discussed in greater detail hereinafter. It is noted that motion assist provided by the resilient element 98, 98′ may provide for the employment of smaller torque motors compared to when the resilient element 98, 98′ is omitted.

Since the lateral motion assembly 61 and the lifting motion assembly 65 each respectively include the first motor 62 and the second motor 66, separate and distinct from one another, the lateral motion assembly 61 can be controlled independently of the lifting motion assembly 65. Independently controlling the first motor 62 and the second motor 66 allows for a variety of variable motion profiles to be selected that include cyclic motion in the first direction, the second direction, or both.

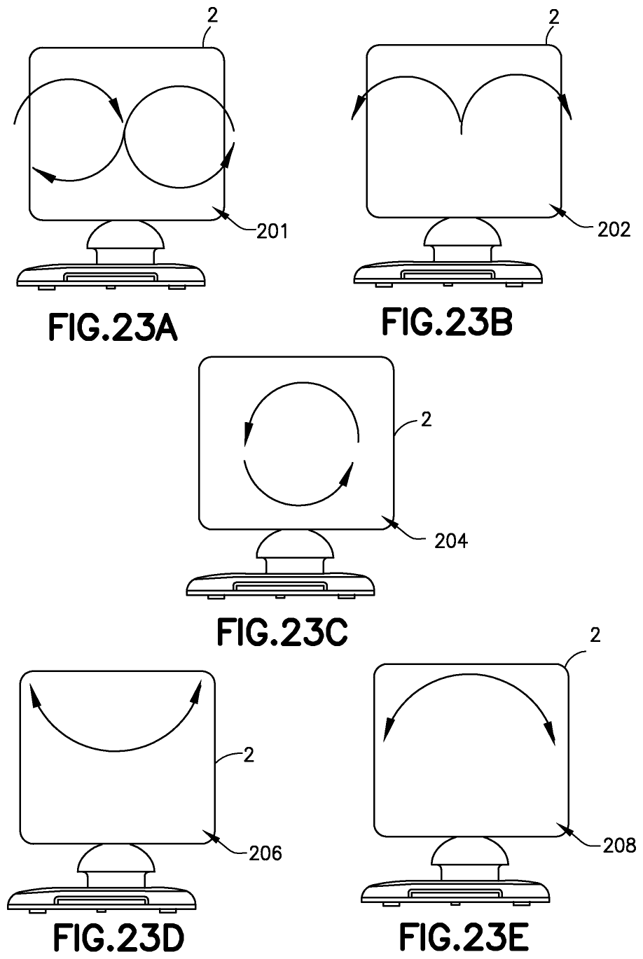

Referring also to FIG. 23A-23E, the control system 50 is configured so as to effect movement of the drive mechanism 60 in at least one motion profile, such as, pre-programmed selectably variable motion profiles Car Ride 201, Kangaroo 202, Ocean Wave 204, Tree Swing 206, and Rock-A-Bye 208, as examples. These selectably variable motion profiles are obtained by independently controlling the horizontal movement provided by the lateral motion assembly 61 and the vertical movement provided by the lifting motion assembly 65 and then coordinating the horizontal and vertical movements to obtain visually distinctive motion profiles. However, these motion profiles are for exemplary purposes only and are not to be construed as limiting as any motion profile including horizontal and/or vertical motions may be utilized. In one aspect, the different selectably variable motion profiles are deterministically defined by a selectably variable velocity characteristic of at least one of the first and second cyclic motions respectively of the first and second motion assemblies 61, 65, and a selectably variable velocity characteristic of at least one of the first and second cyclic motions respectively of the first and second motion assemblies 61, 65. In one aspect, the selectably variable velocity characteristic of at least one of the first and second cyclic motions respectively of the first and second motion assemblies 61, 65, and the selectably variable velocity characteristic of at least one of the first and second cyclic motions respectively of the first and second motion assemblies 61, 65 are selected with the controller 51 from a common selection input to the control system 50.

Referring again to FIGS. 14-22, in one aspect, the vibratory mechanism 90 is coupled to the base 3. In another aspect, the vibratory mechanism 90 is coupled to the movable stage 10 or any other suitable portion of the infant care apparatus 1 (here the vibratory mechanism is mounted to the first platform 70 and positioned to reduce vibratory impulse imparted to the motors 62, 66 of the motion assemblies 61, 65. The vibratory mechanism 90 includes a vibration motor 91 separate and distinct from the first and second motors of the drive mechanism 60. The vibration motor 91 is configured so as to vibrate the movable stage 10. The vibration motor may be any suitable vibration mechanism, such as, a motor with an eccentric weight on the output shaft that rotates about the output shaft to effect vibration. In other aspects, the vibration motor may be any suitable oscillating linear motor or rotary motor. The vibration motor 91 effects vibration in different patterns and intensity so as to form vibration modes which may be selectably imparted on the movable stage 10 as will be discussed in greater detail hereinafter. In one aspect, the vibration profile is superposed over the cyclic motion of the first and/or second motion assembly 61, 65. The vibration profile may be superposed over the lateral motion assembly 61 independent of the lifting motion assembly 65. The vibration profile may be superposed over the lifting motion assembly 65 independent of the lateral motion assembly 61. For example, the vibratory mechanism 90 may be mounted to any stage of the movable stage 10, e.g., to the first platform 70 and/or the support platform 99, to effect a desired vibration superposition. Alternatively, the vibratory mechanism 90 may be mounted to any of the respective driven portions of the lateral motion assembly and/or lifting motion assembly. The stage of the motion assembly to which the vibratory mechanism 90 is attached may be selected freely from concern regarding coupling effecting respective reciprocal motions generated by the corresponding motion assemblies 61, 65. It is noted that the drive mechanism 60, the lifting motion assembly 65, and the vibratory mechanism 90 may be collectively referred to as a drive section of the infant care apparatus 1.

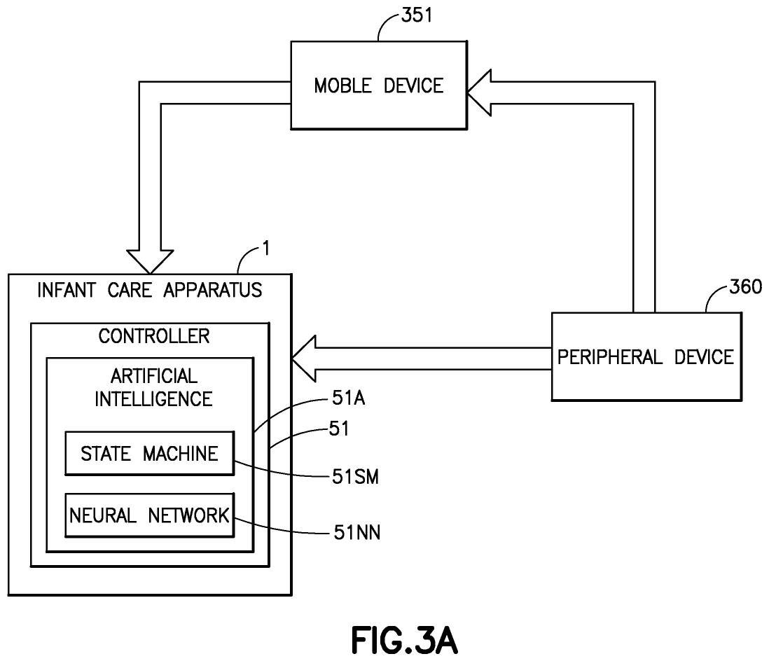

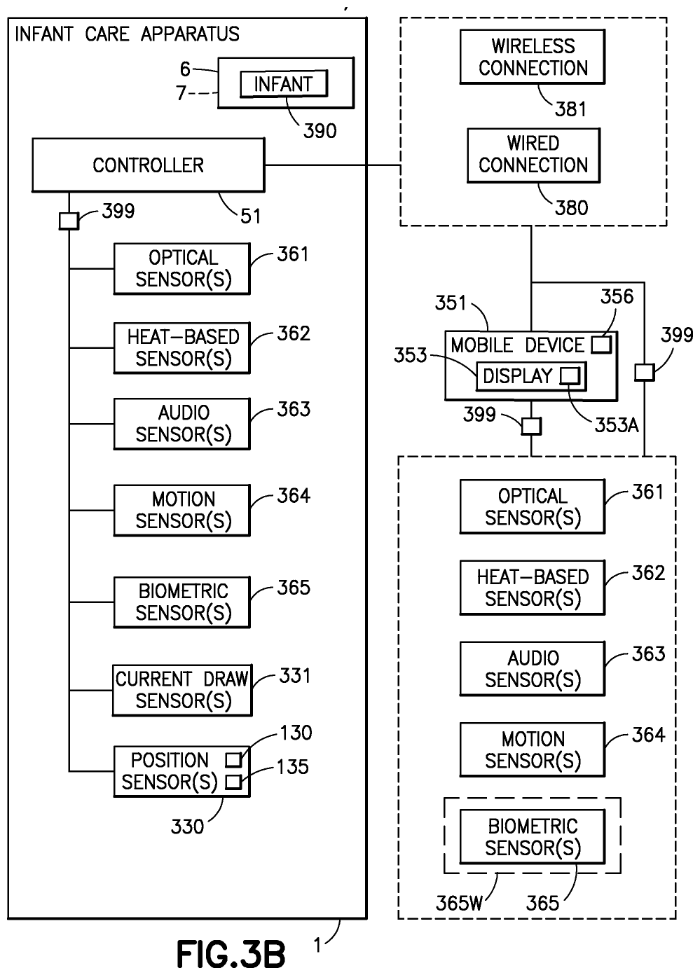

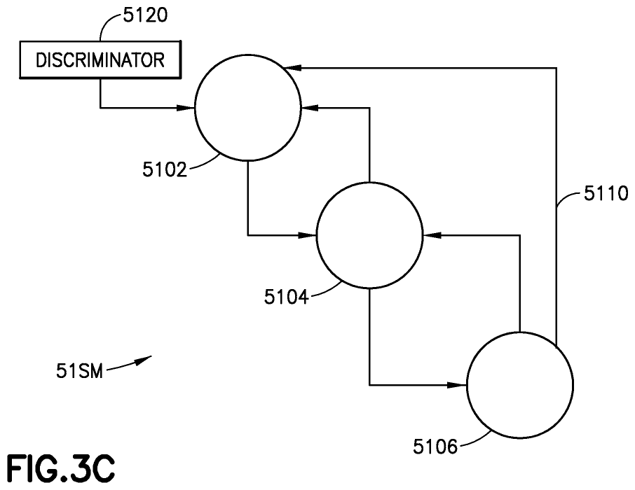

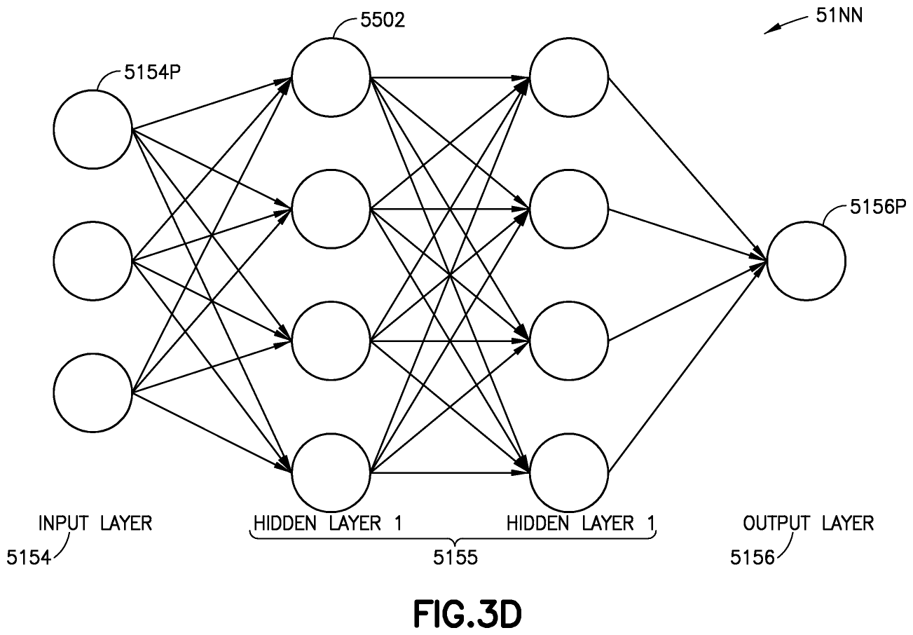

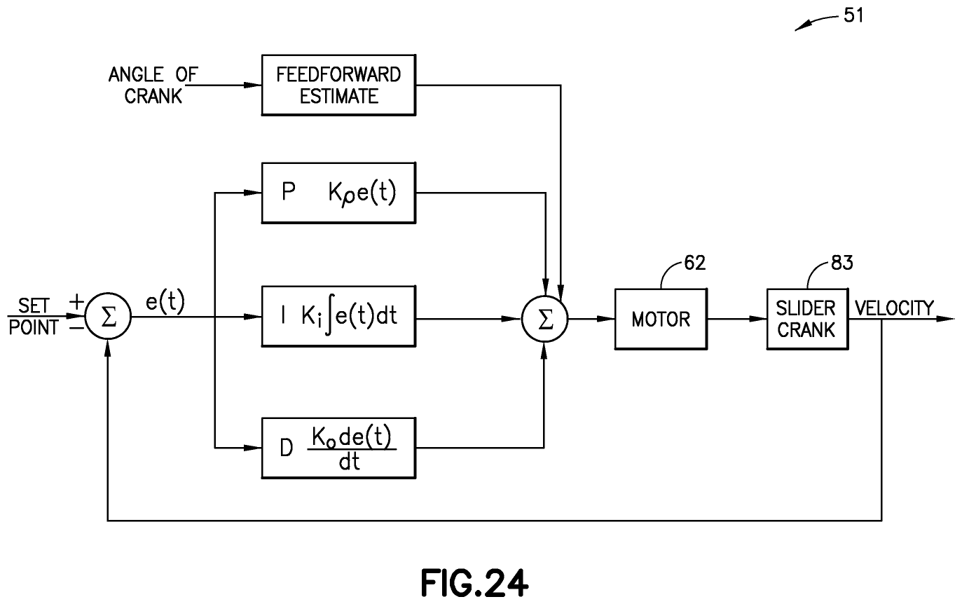

With reference to FIGS. 1, 3A, 3B, 14-22, and 24, the control system 50 may be mounted in the base 3 and provided to effect the different selectably variable motion profiles imparted, by the drive mechanism 60, on the movable stage 10 and to effect, via the vibratory mechanism 90, the various vibration modes for each of the different variable motion profiles. The control system 50 may include any suitable controller 51, such as a microprocessor, a rheostat, a potentiometer, or any other suitable control mechanism to control movement of the drive mechanism 60. In one or more aspects, the controller 51, as will be described in greater detail below is configured with artificial intelligence and includes one or more of a state machine 51SM (FIG. 3C), a neural network 51NN (FIG. 3D), or any other suitable form of artificial intelligence for operating the infant care apparatus 1 in the manner described herein (FIG. 3A). In one or more aspects, the control system 50 includes a remote controller such as a fungible mobile device 351 (FIGS. 3A and 3B) that is communicably connected to the controller 51 through any suitable wired connection 380 or wireless connection 381 (FIG. 3B). In other aspects, the fungible mobile device 351 assumes one or more processing abilities of the controller 51 so that processing capabilities of a portion of the control system 50 onboard the base are supplemented by to completely assumed by the fungible mobile device 351. The mobile device 51 is configured to one or more of display an operational status of the infant care apparatus 1, display a change in operation status of the infant care apparatus 1, display (e.g., with the use of one or more peripheral device 360) a sensed active characteristic of an infant within the infant bed 6 or within the infant seat 7 (generically referred to herein as an infant holder), provide controls to adjust an operation status of the infant care apparatus 1, and/or provide any other suitable operational functionality of the infant care apparatus 1 in a remotely operated manner. The controller 51 with the one or more peripheral device 360 form a closed loop control system that is coupled to the drive section of the infant care apparatus 1 and is configured to adjust actions and/or motions of the infant care apparatus 1 so as to sooth and calm an infant 390 within the infant bed 6 (or infant seat 7).

As noted above, the controller 51 is communicably coupled to the drive mechanism 60 and the vibratory mechanism 90. The controller 51 is configured so as to effect movement of the infant support 2 in the selectably variable motion profiles with selectable vibration modes selected, with the controller, from different selectably variable motion profiles and selectably different vibration modes for each of the different selectable variable motion profiles.

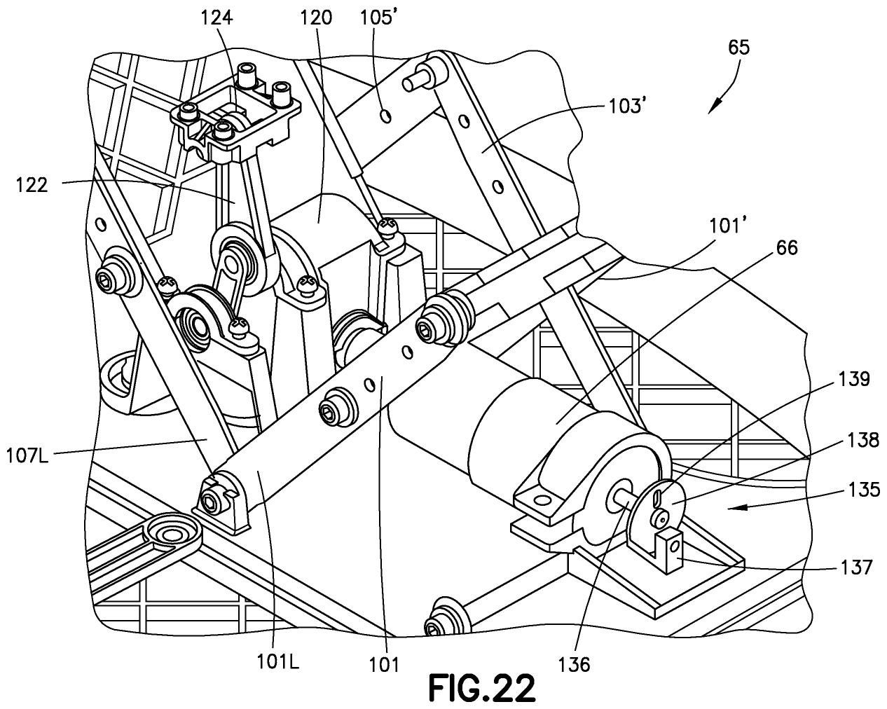

The control system 50 may further include a control panel 52 for viewing and controlling speed and motion of the drive mechanism 60, one or more control switches or knobs 54 for causing actuation of the drive mechanism 60, and a variety of inputs and outputs operatively coupled to the controller 51. For example, the control system 50 may include a horizontal encoder 130 (FIG. 20) coupled to an output shaft 131 of the first motor 62. The horizontal encoder 130 may include an infrared (IR) sensor 132 and a disk 133 with a single hole or slot 134 positioned thereon (see FIG. 20). The horizontal encoder 130 is configured so that the controller 51 may determine the speed and number of revolutions of the first motor 62. A vertical encoder 135 (FIG. 22) may be provided and coupled to a back shaft 136 of the second motor 66. The vertical encoder 135 may include an IR sensor 137 and a disk 138 with a single hole or slot 139 positioned thereon (see FIG. 22). The vertical encoder 135 is configured so that the controller 51 may determine the speed and number of revolutions of the second motor 66. Position of the vibratory mechanism 90 may be selected as previously described so as to avoid generating noise to the position signal of the encoders 130, 135

In addition, while the horizontal encoder 130 and the vertical encoder 135 were described hereinabove, this is not to be construed as limiting as magnetic encoders, as other types of encoders well known in the art may also be used. It may also be desirable to provide an arrangement in which two or more control switches associated with respective motors are required to both be actuated to effect speed control in the desired direction. Furthermore, while it was described that the horizontal encoder 130 and the vertical encoder 135 only include a single slot, this is not to be construed as limiting as encoders with a plurality of slots may be utilized.

In one aspect, the control system 50 may further include horizontal and vertical limit switches 165, 167 (FIG. 14) to provide inputs to the controller 51. For example, the horizontal and vertical limit switches 165, 167 may be configured to indicate to the controller 51 that the first platform 70 or the support platform 99 has reached an end point of travel. The vertical limit switch 167 may be configured to indicate when the support platform 99 is at a lowest and/or highest vertical position relative to the base 3. The horizontal limit switch 165 may be configured to indicate when the first platform 70 is at a point farthest from a center position, relative to the base 3, to the right and/or left. The horizontal and vertical limit switches 165, 167 are configured so that the control system 50 may determine an initial position of the lateral motion assembly 61 and the lifting motion assembly 65 and to adjust the drive mechanism 60 accordingly. In one aspect, the limit switches 165, 167 may be optical switches or any other suitable switches. Position of the vibratory mechanism may be selected as previously described so as to avoid generating noise to the position signal of the limit switches 165, 167 (prevents errors overdriving motors).

The control panel 52 may also have display 53 to provide information to the user, such as, for example, motion profiles, volume of music being played through speakers 56, and speed of the reciprocation motion, etc. In one aspect, the control panel 52 may be a touch screen control panel, a capacitive control panel, or any suitable user interface configured to receive the common selection input from a user for selecting the different selectably variable motion profiles. Control switches 54 (which may be capacitive switches, areas of a touch screen, toggle switches, buttons, etc.) may include user input switches such as a main power switch, a start/stop switch, a motion increment switch, a motion decrement switch, a speed increment switch, a speed decrement switch, and the like. In one or more aspects, at least a portion of the control panel 52 functionality described herein is provided on the mobile device 360 so that the infant care apparatus 1 can be remotely controlled and/or remotely monitored through the mobile device 360. The controller 51 of the control system 50 may also include a variety of outputs. These outputs include, but are not limited to a Pulse Width Modulation (PWM) for the first motor 62, a PWM for the second motor 66, a display backlight.

The following explanation provides an understanding of an exemplary control system 50 of the infant care apparatus 1. Based on the physical limitations of the first motor 62 and the second motor 66 of the lateral motion assembly 61 and the lifting motion assembly 65, the maximum speed of the first motor 62 may be about a four second period and the maximum speed of the second motor 66 may be about a two second period. Based on these constraints, the following relationships may be established:

TABLE 1

| | | | | | Tree | | Rock-a- | | Ocean | |
|---|---|---|---|---|---|---|---|---|---|---|
| | Car Ride | | Kangaroo | | Swing | | Bye | | Wave | |
| Number of | 2 | | 4 | | 2 | | 2 | | 1 | |
| Vertical | | | | | | | | | | |
| Cycles per | | | | | | | | | | |
| Horizontal | | | | | | | | | | |
| Cycle (n) | | | | | | | | | | |
| Phase | 90 | degrees | 0 | degrees | 180 | degrees | 0 | degrees | 90 | degrees |
| offset (Φ) | | | | | | | | | | |
| Horizontal | 8 | seconds | 12 | seconds | 8 | seconds | 8 | seconds | 8 | seconds |
| period at | | | | | | | | | | |
| min speed | | | | | | | | | | |
| Horizontal | 4 | seconds | 8 | seconds | 4 | seconds | 4 | seconds | 4 | seconds |
| speed at | | | | | | | | | | |
| max speed | | | | | | | | | | |

The speed of the first motor 62 is independently set to a period and a feedback control loop is used to ensure that the first motor 62 remains at a constant speed despite the dynamics of the components of the infant care apparatus 1. As mentioned above, the output of the control system 50 is a PWM signal for the first motor 62. One possible input for the control system is velocity of the first motor 62, which can be observed from the speed of the first motor 62 as observed by the horizontal encoder 130. However, in order to avoid computationally expensive calculations, it is possible to operate in the frequency domain and use the number of processor ticks between ticks of the horizontal encoder 130 as the input variable. This allows the calculations of the controller 51 to be limited to integers rather than manipulating floats. The vibratory mechanism 90 generates vibrations in different modes which are superposed over each variable selectable motion profile controlled as noted.

The physical drive mechanism of the lateral motion assembly 61 is the slide crank assembly 80 which is configured so that the first motor 62 reciprocates the first platform 70 back and forth without the need to change directions. Since the first motor 62 is only required to run in one direction, the effect of backlash is eliminated in the system, thereby removing problems with the horizontal encoder 130 on the back shaft 131 of the first motor 62.

It is known that the natural soothing motions a person uses to calm a baby are a combination of at least two motions that each move in a reciprocating motion that has a smooth acceleration and deceleration such that the extremes of the motion slow to a stop before reversing the motion and are fastest in the middle of the motion. This motion is the same as that generated from a sinusoidal motion generated from the combination of the slide crank assembly 80 and the worm gear drive assembly 120. The slide crank assembly 80 and the worm gear drive assembly 120 are configured so that the driving motors run at a constant rotational speed while the output motion provided to the infant seat 7 slows and speeds up, mimicking the motion of a person soothing a child. These assemblies also configured such that the driving motors run in one direction.

With reference to FIGS. 14 and 20, the torque on the first motor 62 depends on the friction of the entire system (which is dependent on weight) and the angle of the crank member 83. The torque of the first motor 62 is controlled by setting the PWM to a predetermined value based on the desired velocity set by the user. Controller 51 may include feed forward compensation to control the velocity of the first motor 62.

Any of the components shown in FIGS. 14-22 may be set to zero. For example, reasonable accuracy is achieved using only proportional and integral terms where the constants Kₚ and Kᵢ are dependent on the input speed, ignoring the feed forward and derivative terms.

Based on the feedback from the horizontal encoder 130 and the horizontal limit switch 165, the exact position of the first platform 70 (denoted “hPos”) can be determined at any point in its range of motion. Similarly, based on feedback from the vertical encoder 135 and the vertical limit switch 167, the exact position of the support platform 99 (denoted “vPos”) can be determined at any point in its range of motion.

While the control of the first platform 70 is based entirely on velocity, the control of the support platform 99 is based upon both position and velocity. For a given horizontal position (hPos) and a given motion, which dictates the number of vertical cycles per horizontal cycles (n) and phase offset (Φ) as shown in Table 1, the desired vPos can be calculated as follows:

Desired_vPos=hPos×xv2h_ratio×n+Φ (Equation 1)

where v2h_ratio is a constant defined as the number of vertical encoder ticks per cycle divided by the number of horizontal encoder ticks per cycle. Based on the actual vertical position, the amount of error can be calculated as follows:

posErr=vPos-Desired_vPos (Equation 2)

This error term must be correctly scaled to +/−verticalEncoderTicksPerCycle/2.

As an aside, if the direction of motion in Ocean Wave 204 and Car Ride 201 is irrelevant, there are two possibilities for Desired_vPos for each value of hPos and we can base the vertical error term, posErr, on the closer of the two.

The positional error term, posErr, must then be incorporated into a velocity based feedback control loop. Logically, if the vertical axis is behind (posErr<0), velocity should be increased while if the vertical axis is ahead (posErr>0), velocity should be decreased in proportion to the error as follows:

vSP=posErr×Kᵥₚ+vBase (Equation 3)

where vBasw=hSP/n×h2v_ratio (Equation 4) and h2v_ratio is defined as the horizontal ticks per cycle/vertical ticks per cycle.

The above description is for exemplary purposes only as any suitable control scheme may be utilized. As noted previously, different modes of vibrations generated by the vibratory mechanism 90 are superposed over each variable selectable motion profile controlled as noted.

In an exemplary embodiment, the infant care apparatus is configured to reciprocate the seat with a vertical displacement of about 1.5 inches and a horizontal displacement of about 3.0 inches with a vertical displacement frequency range of between about 10 and 40 cycles per minute and a horizontal displacement frequency range of between about 10 and 40 cycles per minute. In another example, the infant care apparatus 1 is configured to reciprocate the seat with a vertical displacement more or less than about 1.5 inches and a horizontal displacement more or less than about 3.0 inches with a vertical displacement frequency range of more or less than about 10 to 40 cycles per minute and a horizontal displacement frequency range of more or less than about 10 to 40 cycles per minute.

In another aspect, at least a third reciprocation means (not shown) may be added to enable reciprocation of the seat in another direction different than the first and second directions imparted by the first and second motion assemblies 61, 65 referenced herein.

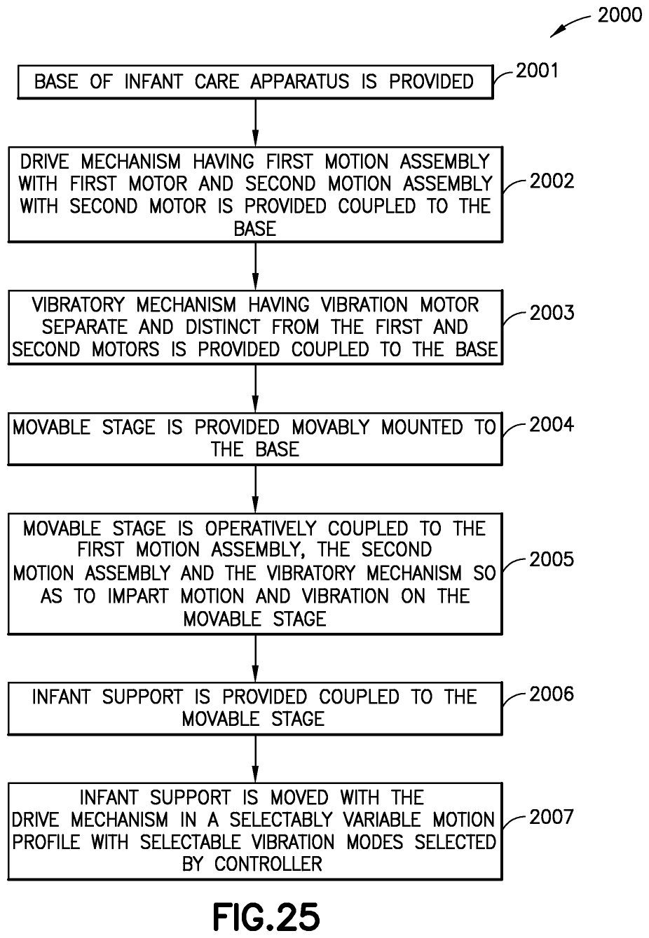

Referring to FIGS. 1, 2, 14-22, and 25, a method 2000 for imparting motion on an infant support 2 is illustrated. The method includes providing base 3 of infant care apparatus 1 (FIG. 25, Block 2001). Drive mechanism 60 having lateral motion assembly 61 and lifting motion assembly 65 is provided coupled to the base 3 (FIG. 25, Block 2002), wherein the lateral motion assembly 61 has first motor 62 dependent from the base 3 and the lifting motion assembly 65 has second motor 66 separate and distinct from the first motor 62. Vibratory mechanism 90 having vibration motor 91 separate and distinct from the first and second motors 62, 66 of the drive mechanism 60 is provided a coupled to the base 3 (FIG. 25, Block 2003). Movable stage 10 is provided movably mounted to the base 3 (FIG. 25, Block 2004). The movable stage 10 is operatively coupled to the lateral motion assembly 61 so that the first motor 62 imparts, via the lateral motion assembly 61, a first cyclic motion in a first direction D1 to the movable stage 10, and to the lifting motion assembly 65 so that the second motor 66 imparts, via the lifting motion assembly 65, a second cyclic motion to at least part of the movable stage 10 in a second direction D2 independent of the first cyclic motion in the first direction D1 imparted by the lateral motion assembly 61 and to the vibratory mechanism 90 so that the vibration motor 91 vibrates the movable stage 10 (FIG. 25, Block 2005). Infant support 2 is provided coupled to the movable stage 10 (FIG. 25, Block 2006) so that the second cyclic motion and first cyclic motion is imparted to the infant support 2, and the infant support is configured to move cyclically in both the first direction D1 and the second direction D2 relative to the base 3. Controller 51 communicably coupled to the drive mechanism 60, moves the infant support 2 in a selectably variable motion profile with selectable vibration modes selected, with the controller 51, from different selectably variable motion profiles and selectably different vibration modes for each of the different selectable variable motion profiles (FIG. 25, Block 2007).

Referring again to FIGS. 3A and 3B, as noted above, the control system 50 includes one or more peripheral device 360 that is/are communicably coupled to the controller 51 through any suitable wired connection 380 or wireless connection 381. The one or more peripheral device 360 is configured to observe at least one characteristic of an infant in the infant bed 6 (or infant seat 7). The one or more peripheral device 360 may be integral with the infant care apparatus 1 (such as integrated into the infant bed 6 or base 3) or be remotely connected to the infant care apparatus 1 so as to form a remote accessory device of the infant care apparatus 1.

Data from the one or more peripheral device 360 is registered by the controller 51 (or by the mobile device 351 so as to effect through the controller 51) and employed to provide infant stimulation to calm and sooth the infant's mood (e.g., stop the infant from crying) or state (e.g., cause/help the infant to fall asleep and/or reduce restlessness of the infant). For example, the data from the one or more peripheral devices can be employed (as will describe herein) by the controller 51 to initiate one or more of the predetermined motions (as described herein) of the infant bed 6, initiate one or more sounds (and select a suitable sound volume), activate lights, and/or initiate vibration (as described herein) of the infant bed 6. The predetermined motions, sounds, changes in sound volume, lights, and vibrations can be activated by the controller 51 singularly or in parallel for overlapping stimulation of the infant. As noted above, the controller 51 in one or more aspects includes artificial intelligence 51A where the controller 51 is configured to vary and adjust the stimulation types and stimulation intensities provided to the infant based on the effectiveness of chosen stimulants (e.g., as determined by peripheral device feedback), so that over time the controller 51 becomes more effective at (or learns how to) sooth and entertain the needs of the infant within the infant bed 6. It is noted that while the control system 50 is described herein with respect to the infant bed 6, the infant seat 7 may be controlled in a similar manner.

As non-limiting examples of the one or more peripheral device 360, the one or more peripheral device 360 includes optical sensor(s) 361, heat-based sensor(s) 362, audio sensor(s) 363, motion sensor(s) 364, biometric sensor(s) 365, and/or any other suitable sensor(s) configured to observe (or otherwise monitor) the at least one characteristic of the infant. The optical sensor(s) 361 are any suitable optical sensors including, but not limited to, one or more of CCD and CMOS cameras. The heat-based sensor(s) 362 may be any suitable heat-based sensors including, but not limited to, any suitable pyroelectric sensors such as infrared sensors. The optical sensor 361 and heat-based sensor 362 are employed by the controller 51 (and/or mobile device 351) to monitor and measure, in conjunction with the controller 51 one or more of a change in position of the infant within the infant bed 6 (e.g., where the infant position in a series of video frames is compared to determine infant motion), infant facial expression, and/or blood oxygenation level and pulse of the infant such as through image recognition using video of the infants skin tone/color.

The audio sensor(s) 363 are any suitable sensors for detecting and/or otherwise measuring sound waves including, but not limited to, any suitable microphones. The audio sensor(s) 363 are employed by the controller 51 (and/or mobile device 351) to measure or otherwise detect changes in sound compared to nominal background noise (i.e., ambient noise of the environment in which the infant are apparatus 1 is located). The audio sensor(s) 363 are also employed by the controller 51 (and/or mobile device 351) to measure or otherwise detect types of noises from the infant within the infant bed 6, where the infant noises include, but are not limited to, crying, cooing, and babbling.

The motion sensor(s) 364 are any suitable sensors for detecting and/or otherwise measuring motion/movement of the infant within the infant bed 6. The motion sensor(s) 364 include but are not limited to one or more of cameras (e.g., to detect motion through frame comparison and image recognition), accelerometers, gyroscopes, inertial measurement units (IMUS), piezoelectric sensors, air pressure sensors, electromotive force (EMF) sensors, or any other suitable motion sensor. The motion sensor(s) 364 are employed by the controller 51 (and/or mobile device 351) to measure or otherwise detect motion of the infant within the infant bed 6 where the motion sensors are located on or worn by the infant and/or are located adjacent to (but not worn by or located on) the infant within the infant bed 6.

The biometric sensor(s) 365 are any suitable sensors for detecting and/or otherwise measuring biometrics of the infant within the infant bed 6. The biometric sensor(s) 365 include, but are not limited to, blood pressure monitors, heart rate monitors, thermometers, skin conductance sensors, pulse oximeter, motion sensors (e.g., accelerometers, gyroscopes, inertial measurement units (IMUs)) and/or any other suitable biometric sensor. In one or more aspects, the biometric sensor(s) 265 are wearable health devices 365W (FIG. 3B) (e.g., and may be referred to as “smart” wearables) such as, for example, cuffs, wrist bands, hats, pants, shirts, anklets, socks, undergarments, diapers, etc. that include one or more of the biometric sensor(s) 365 described above. The biometric sensors are, in one aspect, removable from the smart wearables and/or interchangeable from one smart wearable to another smart wearable to facilitate washing or disposal (such as when used with a disposable “smart” diaper) of the smart wearable; while in other aspects, the biometric sensors are non-removable and substantially environment proof (again to facilitate washing of the smart wearable). The biometric sensor(s) are employed by the controller 51 (and/or mobile device 351) to measure or otherwise detect biometrics such as, for example, blood oxygen levels, pulse rate, body temperature, and skin conductivity.

In addition to, or in lieu of, one or more of the above-noted peripheral devices 360, the infant care apparatus 1 includes position sensors 330 (such as the encoders described above) and/or current draw sensors 331. The current draw sensors 331 are communicably coupled to the drive system motors (such as at least motors 62, 66) and are configured to monitor current used by the motor(s) of the drive system. The controller 51 (or mobile device 351) is configured to determine the state (e.g., such as restlessness) of the infant within the infant bed 6 by comparing the current and positional information of the infant bed 6 (or infant seat 7) with an active infant therein with the current and position information of the infant bed 6 (or infant seat 7) with a non-active infant therein.

As noted above, data from the one or more peripheral device 360 is registered by the controller 51 (or by the mobile device 351 so as to effect through the controller 51) and employed by the controller 51 to effect a change in one or more of the action (e.g., aural stimulus (sounds, music, etc.) and visual stimulus (lights, the mobile 19, etc.)) and the motion (vibratory and motion profiles) of the infant bed 6. The change in the one or more of the action and the motion of the infant bed 6 provides infant stimulation to calm and sooth the infant's mood or state. Each of the above noted peripheral devices 360, 361-365 and sensors 330, 331 (collectively referred to herein as sensors) is configured to generate a sensor signal 399 that embodies at least one characteristic of the infant within the infant bed 6, which characteristics include those measured by the peripheral devices 360, 361-365 and sensors 330, 331. The at least one characteristic is an action characteristic (e.g., crying, cooing, babbling, and/or other aural action) and/or a motion characteristic (e.g., kicking, rolling, and/or other movements) of the infant 390. It is noted that in one or more aspects the sensors communicate directly with the controller 51, while in other aspects the sensors communicate with the controller 51 indirectly such as through a mobile device 351 (See FIG. 3B). The controller 51 is configured to send one or more commands to the drive section to activate or drive the motors 62, 66 (and/or vibratory mechanism 90) in a predetermined sequence based on the sensor data of the at least one characteristic sensed by the peripheral devices 360, 361-365 and sensors 330, 331. The one or more commands are, in one or more aspects, a responsive change (generated by the controller 51) in the one or more of the action and the motion of the infant bed 6 based on a change in one or more of the action characteristic and motion characteristic of the infant 390, which characteristics are sensed by the peripheral devices 360, 361-365 and sensors 330, 331.

As noted above and referring also to FIGS. 3C and 3D, in one or more aspects, the controller 51 includes artificial intelligence 51A. The artificial intelligence 51A is configured to monitor the actions and motions of the infant 390 (i.e., through the sensors coupled to the controller 51) and the controller's responsive changes generated in response thereto. The artificial intelligence 51A also monitors (e.g., through the sensors coupled to the controller 51, such as the biometric sensors of the smart wearables or other suitable sensors as described herein) the infant's 390 reactions to the responsive changes so that, over time, the controller 51 learns how the infant 390 responds to the reactive changes so as to better (i.e., more effectively) suit the soothing and calming of the infant 390. The artificial intelligence 51A, is in one or more aspects, configured to predict, using active learning (as described herein) and input from one or more of the sensors described herein, a type of change event that triggers a change in the infant's biometrics (e.g., the reason(s) causing the infant to become fussy, irritated, etc., where the change event(s) include hunger, a soiled diaper/clothing (e.g., a urination or defecation event), environmental conditions, illness, etc.).

FIG. 3C illustrates a simple example of a finite state machine 51SM, but it should be understood that states machines employed for real-time control of the infant care apparatus 1 may be more complex, having more states and transitions than illustrated in FIG. 3C. The state machine 51SM includes any suitable number of states such as a first state 5102, a second state 5104, and a third state 5106. The states represent, for example, states (i.e., crying, cooing, babbling, sleeping, awake, restless, temperature, fever, chills, or any other suitable state) of the infant 390 within the infant holder. Each change from one state to another state occurs through a transition such as a first transition 5110. It is noted that each state may have more than one transition into or out of that state. While a simple finite state machine 51SM is illustrated in FIG. 3C, the state machine may be any suitable state machine including but not limited to state machines that are fully represented by a state table that relates states and conditions in tabular form. In addition, the state machine 51MS employs any suitable models for state transitions. For example, certain state machine models define binary conditions for transitions while other state machine models permit more generalized expressions for evaluating state changes (for example, a Moore state machine has output that depend only on the current state, and a Mealy state machine has outputs that depend on an input and the state). Other suitable state machines include algorithmic state machines, Unified Modeling Language state diagrams, directed graphs, etc.

The state machine 51SM includes or otherwise forms a discriminator 5120 that discriminates between the different input from the peripheral devices 360, 361-365 and sensors 330, 331 and, in one or more aspects, further discriminates the intensity of such inputs so that the state machine 51SM provides an output 5106 that corresponds with the inputs and, in some aspects the intensity of the inputs. For example, the state machine discriminates the input intensity by applying one or more thresholds to one or a combination of inputs from the peripheral devices 360, 361-365 and sensors 330, 331. Examples of thresholds with which the discriminator is configured include but are not limited to, temporal thresholds, intensity thresholds, and input combination thresholds.

One example of a temporal threshold that is applied by the state machine 51SM is a duration of infant activity before the state machine 51SM provides an output that modifies one or more of the action and motion of the infant care device 1. As an example, where the infant 390 is crying and a motion of the infant car device is set to the car ride motion 201, the discriminator 5120 discriminates whether the crying has been stopped for any suitable predetermined time before the state machine provides an output 5156P that modifies or stops the motion of the infant care apparatus 1.

One example of an intensity threshold that is applied by the state machine 51SM is where thresholds are applied to a heart rate of the infant (e.g., depending on the infant's age) for adjusting one or more of the action and motion of the infant care device. For example, the discriminator 5120 discriminates the intensity of the infant's heartbeat with respect to one or more thresholds. A first or upper threshold may be indicative of a high degree of restlessness of the infant 390. A second or intermediate threshold may be indicative of a moderate degree of restlessness. A heartbeat below the second threshold may be indicative of substantially no restlessness. Where the discriminator determines the infant's 390 heartbeat is above the first threshold the state machine 51SM outputs commands to, for example, the drive system so that the infant holder moves in one of the movement patterns described herein (which movement patterns may also be determined by the discriminator) with a first speed that corresponds to the first threshold. Where the discriminator determines the infant's 390 heartbeat is above the second threshold but below the first threshold the state machine 51SM outputs commands to, for example, the drive system so that the infant holder moves in one of the movement patterns described herein (which movement patterns may also be determined by the discriminator) with a second speed that corresponds to the second threshold. Where the discriminator determines the infant's 390 heartbeat to be below the second threshold the state machine 51SM outputs commands to, for example, the drive system so that the infant holder moves in one of the movement patterns described herein (which movement patterns may also be determined by the discriminator) with a third speed. Here, the first speed is greater/faster than the second speed, and the second speed is greater/faster than the third speed. It is noted that the third speed may be zero where the infant holder is held substantially stationary.

One example of an input combination threshold is where a predetermined combination of multiple inputs trigger a change one or more of the action and the motion of the infant holder. For example, where the discriminator 5120 determines, from peripheral device/sensor data, that the infant's heart rate is increased but no other input exists (e.g., data from other peripheral devices/sensors is null or nominal) the state machine 51SM may output no change in action or motion of the infant holder. However, where the discriminator 5120 determines, from peripheral device/sensor data, that the infant's heart rate is increased and the infant is crying, the state machine 5120 outputs a change in one or more of the action and motion. For example, the output of the state machine may be to activate the drive section so that the infant holder moves in one of the predetermined motion patterns described herein. The predetermined combination of multiple inputs is adaptable, changing according to registration of changes in sensed characteristics, from a first predetermined combination to an adapted predetermined combination. For example, the controller may apply various heuristic algorithms and/or neural networks to data histories of biometric sensor data to identify corresponding characteristic combinations and/or thresholds for one or more states and revise or change the predetermined combination to an adapted predetermined combination.

While, movement of the infant holder has been described above in response to input to the state machine 51SM, such response generated by the state machine 51SM is not limited to movement of the infant holder. For example, the state machine may output command(s) that initiate one of or any suitable combination of audible sounds (e.g., music, white noise, etc.), movement of the infant holder in predetermined patterns, and vibration of the infant holder.

FIG. 3D illustrates a generalized neural network 51NN for illustrative purposes only and it should be understood that the size and depth of the neural network 51NN employed to control the infant care apparatus 1 may vary from that shown in FIG. 3D. For illustrative purposes only the neural network may include, for example, a two-layer network of objects 5502 in a middle layer 5155 of the neural network 51NN. An input 5154P may be applied to the objects 5502 at an input layer 5154 of the neural network 51NN and an output 5156P may be produced at an output layer 5156. Each of the objects 5502 in the exemplary neural network 51NN contains any number of artificial neurons and objects. The neural network is trained using any suitable criteria (such as training data obtained from any number of infants within an infant holder, such as described herein, or from any other source such as clinically known biometrics of infants). The neural network is configured to learn, over time, the particular characteristics of the infant (e.g., of the end user) so that outputs of the neural network 51NN improve over time with respect to soothing the infant within the infant holder. The neural network may output command(s) that initiate (with any suitable intensity as determined by the neural network) one of or any suitable combination of audible sounds (e.g., music, white noise, etc.), movement of the infant holder in predetermined patterns, and vibration of the infant holder. As noted above, the neural network, is in one or more aspects, configured to predict, using active learning and input from one or more of the sensors described herein, the change event that causes the infant's biometrics to change. Here the neural network may be trained with any suitable training data that is indicative of one or more of the change events noted above so as to predict future occurrences of the one or more change events. The neural network, in one or more aspects, may also receive feedback (such as through a mobile device 351, the control panel or any other suitable user interface in communication with the controller 51 and neural network thereof) from a parent or guardian of the infant after an occurrence of one or more of the change events to facilitate active learning by the neural network. A notification of the type of change event that has or is occurring may be presented on any suitable display of the mobile device and/or control panel (or any other suitable user interface in communication with the controller 51 and neural network thereof) along with a notification of a change in biometrics.

Still referring to FIGS. 1, 3A, and 3B, the control panel 52 includes any suitable display (such as display 53) configured to provide any suitable indicia 353A to a user (e.g., caretaker) of the infant care apparatus 1. In one or more aspects, the mobile device 351 also includes any suitable display 353 configured to provide the indicia 353A to a user of the infant care apparatus 1, in lieu of viewing the indicia on the control panel 52. The controller 51 is configured to generate the indicia 353A (or cause an illumination or display of the indicia) to identify the responsive change in the one or more of the action (e.g., aural stimulus and visual stimulus) and the motion (e.g., vibratory and motion profiles) of the infant seat 6. For exemplary purposes only, where the controller 51 activates the vibratory mechanism 90 or changes a motion profile of the infant bed 6 in response to sensed characteristics of the infant 390, the indicia 353A presented to the user indicates that the vibratory mechanism 90 has been activated (i.e., vibratory mechanism has been turned on) or that the motion profile has changed (i.e., motion profile changed to “car ride”). In one or more aspects the indicia indicates both the prior state (i.e., the “from” state) and the current state (i.e., the “to” state) of the action and/or motion such as where, for exemplary purposes only, the motion profile was changed from “car ride” to “tree swing” or sound emitted from the speakers 56 was changed from “music” to “white noise”. The indicia 353A is in one aspect a visual indicia presented on the display 53 while in other aspects the indicia is presented as an aural indicia (e.g., spoken words or other suitable audible tones or phonetics) through any suitable speakers, such as speakers 56 of the infant care device or speakers 356 of the mobile device 351. In still other aspects, the indicia 353A are presented as both aural indicia and visual indicia.

In one or more aspects, the controller 51 is configured to generate the indicia 353A (or cause an illumination or display of the indicia) where the indicia 353A identifies the change in one or more of the action characteristic or the infant 390 and the motion characteristic of the infant 390 sensed by the sensors (e.g., the peripheral devices 360, 361-365 and sensors 330, 331). For exemplary purposes only, where the controller 51 receives sensor signals 399 that embody a change in sensed characteristics of the infant 390, the indicia 353A presented to the user indicates that the sensed characteristic has changed. For example, the sensed characteristic may be a motion characteristic where motion characteristic changed from restless movement to substantially motionless (e.g., sleeping). Here, the controller 51 generates the indicia to indicate the motion characteristic has changed to substantially motionless. As another example, the sensed characteristic is a temperature of the infant 390, where the temperature rises from 98.6° F. (37° C.) to 100° F. (37.8° C.). Here the controller 51 generates the indicia to indicate that the temperature of the infant 390 has risen (and in some aspects the indicia includes a numerical temperature). In one or more aspects the indicia indicates both the prior state (i.e., the “from” state) and the current state (i.e., the “to” state) of the action and/or motion characteristic of the infant 390 where, for exemplary purposes only, the temperature of the infant changed from “98.6° F. (37° C.)” to “100° F. (37.8° C)”. As noted above, the indicia 353A is in one aspect a visual indicia presented on the display 53 while in other aspects the indicia is presented as an aural indicia (e.g., spoken words or other suitable audible tones or phonetics) through any suitable speakers, such as speakers 56 of the infant care device or speakers 356 of the mobile device 351. In still other aspects, the indicia 353A are presented as both aural indicia and visual indicia.

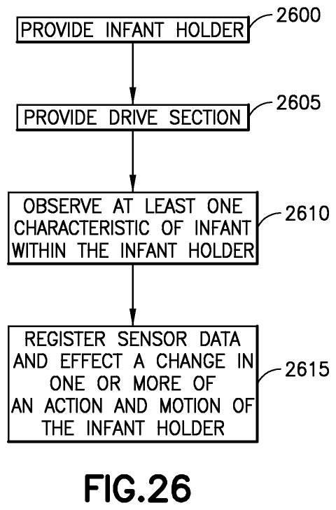

Referring to FIGS. 1, 3A, 3B, and 26, an exemplary method of operating the infant care apparatus 1 will be described. In accordance with the method, an infant holder (such as the infant bed 6 or infant seat 7) is provided (FIG. 26, Block 2600). The drive section (as noted above which includes the drive mechanism 60, the lifting motion assembly 65, and the vibratory mechanism 90) is provided (FIG. 26, Block 2605), where the drive section is coupled to the infant holder and has a motor (e.g., one or more of motors 62, 66 and vibratory mechanism 90) configured to generate the one or more of the action and the motion of the infant holder. At least one characteristic of the infant 390 is observed, with the sensors (e.g., peripheral devices 360, 361-365 and sensors 330, 331) (FIG. 26, Block 2610). The controller 51 registers sensor data from the sensors and effects a change in the one or more of the action and the motion of the infant holder (FIG. 26, Block 2615). As described above, the controller 51 sends a command(s) to the motor (e.g., one or more of motors 62, 66 and vibratory mechanism 90) based on the sensor data of the at least one characteristic sensed by the peripheral devices 360, 361-365 and sensors 330, 331. As also described above, the controller 51 generates an aural and/or visual indicia to identify one or more of an action characteristic of the infant 390 sensed by the sensors, a motion characteristic of the infant 390 sensed by the sensor, a change in the action characteristic of the infant 390 sensed by the sensor, and a change in the motion characteristic of the infant 390 sensed by the sensor.

In accordance with one or more aspects of the disclosed embodiment an infant apparatus having an infant support is provided. The infant apparatus including a base, and an infant support coupling arranged so as to releasably couple the infant support to the base, the infant support coupling including a movable support movably connect to the base and disposed so as to form a support seat that engages and supports the infant support on the base, with the movable support in a first position (relative to the base), and actuable grip members configured to actuate between a closed position and an open position to capture and release the infant support to the base, the actuable grip members being automatically actuable between the closed and open positions by action of the movable support moving to the first position.

In accordance with one or more aspects of the disclosed embodiment the actuable grip members are disposed with respect to the infant support to effect grip.

In accordance with one or more aspects of the disclosed embodiment the infant support is free of grip.

In accordance with one or more aspects of the disclosed embodiment movable support has cams that cam grip members from closed to/from open position.

In accordance with one or more aspects of the disclosed embodiment an infant care apparatus is provided. The infant care apparatus including a base, a drive mechanism coupled to the base and having a first motion assembly and a second motion assembly, wherein the first motion assembly has a first motor dependent from the base and the second motion assembly has a second motor separate and distinct from the first motor, a vibratory mechanism coupled to the base, the vibratory mechanism having a vibration motor separate and distinct from the first and second motors of the drive mechanism, a movable stage movably mounted to the base and operatively coupled to the first motion assembly so that the first motor imparts, via the first motion assembly, a first cyclic motion in a first direction to the movable stage, and to the second motion assembly so that the second motor imparts, via the second motion assembly, a second cyclic motion to at least part of the movable stage in a second direction independent of the first cyclic motion in the first direction imparted by the first motion assembly and to the vibratory mechanism so that the vibration motor vibrates the movable stage, an infant support coupled to the movable stage so that the second cyclic motion and first cyclic motion is imparted to the infant support, and the infant support is configured to move cyclically in both the first direction and the second direction relative to the base, and a controller communicably coupled to the drive mechanism, and configured so as to move the infant support in a selectably variable motion profile with selectable vibration modes selected, with the controller, from different selectably variable motion profiles and selectably different vibration modes for each of the different selectable variable motion profiles.

In accordance with one or more aspects of the disclosed embodiment the controller is configured to configured so as to move the infant support with separate impetus separately imparted on the infant support by the first cyclic motion and second cyclic motion respectively driven by the first and second motors, in both the first direction and the second direction with the selectably variable motion profile.

In accordance with one or more aspects of the disclosed embodiment the controller is configured to effect selection of the selectably variable motion profile by separate variance of motion characteristic of the separate respective first cyclic motion and second cyclic motion determined from a common selection input to the controller selecting the selectably variable motion profile

In accordance with one or more aspects of the disclosed embodiment at least part of the movable stage isolates the drive mechanism from the base.

In accordance with one or more aspects of the disclosed embodiment each of the different selectably variable motion profiles is deterministically defined by a selectably variable velocity characteristic of at least one of the first and second cyclic motions respectively of the first and second motion assemblies, and a selectably variable velocity characteristic of at least one of the first and second cyclic motions respectively of the first and second motion assemblies.

In accordance with one or more aspects of the disclosed embodiment the selectably variable velocity characteristic of at least one of the first and second cyclic motions respectively of the first and second motion assemblies, and the selectably variable velocity characteristic of at least one of the first and second cyclic motions respectively of the first and second motion assemblies are selected with the controller from the common selection input to the controller.

In accordance with one or more aspects of the disclosed embodiment each of the different selectably variable motion profiles includes at least one of horizontal and vertical movements.

In accordance with one or more aspects of the disclosed embodiment the first motion assembly includes the first motor having a drive shaft, and a slide crank assembly comprising a gearing assembly coupled to the drive shaft of the first motor and a crank member coupled to the gearing assembly and the movable stage, wherein operation of the first motor causes rotation of the slide crank assembly, thereby imparting the first cyclic motion to the movable stage.

In accordance with one or more aspects of the disclosed embodiment the second motion assembly includes the second motor having a drive shaft, a worm gear assembly coupled to the output of the drive shaft, and a vertical yoke having a first end coupled to an output shaft of the worm gear assembly, wherein operation of the second motor causes rotation of the vertical yoke, thereby imparting second cyclic motion to the infant support.

In accordance with one or more aspects of the disclosed embodiment the second motion assembly further includes a dual scissor mechanism coupled to a second end of the vertical yoke configured to support the infant support.

In accordance with one or more aspects of the disclosed embodiment a first encoder having a single slot is coupled to a first drive shaft of the first motor and a second encoder having a single slot is coupled to a second drive shaft of the second motor.

In accordance with one or more aspects of the disclosed embodiment the controller determines position information of the infant support based at least in part on information from the first encoder and the second encoder.

In accordance with one or more aspects of the disclosed embodiment a method is provided. The method including providing a base of an infant care apparatus, providing a drive mechanism coupled to the base, the drive mechanism having a first motion assembly and a second motion assembly, wherein the first motion assembly has a first motor dependent from the base and the second motion assembly has a second motor separate and distinct from the first motor, providing a vibratory mechanism coupled to the base, the vibratory mechanism having a vibration motor separate and distinct from the first and second motors of the drive mechanism, providing a movable stage movably mounted to the base and operatively coupled to the first motion assembly so that the first motor imparts, via the first motion assembly, a first cyclic motion in a first direction to the movable stage, and to the second motion assembly so that the second motor imparts, via the second motion assembly, a second cyclic motion to at least part of the movable stage in a second direction independent of the first cyclic motion in the first direction imparted by the first motion assembly and to the vibratory mechanism so that the vibration motor vibrates the movable stage, providing an infant support coupled to the movable stage so that the second cyclic motion and first cyclic motion is imparted to the infant support, and the infant support is configured to move cyclically in both the first direction and the second direction relative to the base, and moving, with a controller communicably coupled to the drive mechanism, the infant support in a selectably variable motion profile with selectable vibration modes selected, with the controller, from different selectably variable motion profiles and selectably different vibration modes for each of the different selectable variable motion profiles.

In accordance with one or more aspects of the disclosed embodiment a first encoder is coupled to a first drive shaft of the first motor and a second encoder is coupled to a second drive shaft of the second motor.

In accordance with one or more aspects of the disclosed embodiment the first encoder and the second encoder each include no more than one slot.

In accordance with one or more aspects of the disclosed embodiment determining, with the controller, position information of the infant support based at least in part on information from the first encoder and the second encoder.

In accordance with one or more aspects of the disclosed embodiment each of the different selectably variable motion profiles is predetermined, the method further comprising selecting, by a user, one of the selectably variable motion profiles.

In accordance with one or more aspects of the disclosed embodiment an infant care apparatus comprises an infant holder; a drive section coupled to the infant holder and having a motor configured to generate one or more of an action and a motion of the infant holder; a biometric sensor configured to observe at least one characteristic of an infant within the infant holder; and a controller configured to employ a neural network or state machine that is communicably coupled to the biometric sensor and the drive section, where the controller registers sensor data from the biometric sensor and effects, through the neural network or the state machine, a change in the one or more of the action and the motion of the infant holder.

In accordance with one or more aspects of the disclosed embodiment the biometric sensor is configured to generate a sensor signal embodying the at least one characteristic of the infant, where the at least one characteristic is one or more of an action characteristic of the infant and a motion characteristic of the infant.

In accordance with one or more aspects of the disclosed embodiment the controller is configured to send a command to the motor based on the sensor data of the at least one characteristic sensed by the sensor.

In accordance with one or more aspects of the disclosed embodiment the controller generates a responsive change in the one or more of the action and the motion of the infant holder based on a change in one or more of an action characteristic of the infant and a motion characteristic of the infant sensed by the biometric sensor.

In accordance with one or more aspects of the disclosed embodiment the controller is configured to generate indicia to identify the responsive change in the one or more of the action and the motion of the infant holder.

In accordance with one or more aspects of the disclosed embodiment the indicia is one or more of aural indicia and visual indicia.

In accordance with one or more aspects of the disclosed embodiment the controller is configured to generate indicia to identify the change in one or more of the action characteristic of the infant and the motion characteristic of the infant sensed by the biometric sensor.

In accordance with one or more aspects of the disclosed embodiment the indicia is one or more of aural indicia and visual indicia.

In accordance with one or more aspects of the disclosed embodiment the biometric sensor comprises one or more of a vision sensor, a heat-based sensor, an audio sensor, a motion sensor, and a biometric sensor.

In accordance with one or more aspects of the disclosed embodiment the biometric sensor comprises a wearable health device.

In accordance with one or more aspects of the disclosed embodiment the drive section is configured to provide more than one degree of freedom motion to the infant holder.

In accordance with one or more aspects of the disclosed embodiment a method for infant care with an infant care apparatus, the method comprises providing an infant holder; providing a drive section coupled to the infant holder and having a motor configured to generate one or more of an action and a motion of the infant holder; observing, with a biometric sensor, at least one characteristic of an infant within the infant holder; and registering, with a controller configured for employing a neural network or state machine, sensor data from the sensor and effecting, with the neural network or state machine, a change in the one or more of the action and the motion of the infant holder.

In accordance with one or more aspects of the disclosed embodiment the biometric sensor generates a sensor signal embodying the at least one characteristic of the infant, where the at least one characteristic is one or more of an action characteristic of the infant and a motion characteristic of the infant.

In accordance with one or more aspects of the disclosed embodiment the controller sends a command to the motor based on the sensor data of the at least one characteristic sensed by the biometric sensor.

In accordance with one or more aspects of the disclosed embodiment the controller generates a responsive change in the one or more of the action and the motion of the infant holder based on a change in one or more of an action characteristic of the infant and a motion characteristic of the infant sensed by the biometric sensor.

In accordance with one or more aspects of the disclosed embodiment the controller generates indicia to identify the responsive change in the one or more of the action and the motion of the infant holder.

In accordance with one or more aspects of the disclosed embodiment the controller is configured to generate indicia to identify the change in one or more of the action characteristic of the infant and the motion characteristic of the infant sensed by the biometric sensor.

In accordance with one or more aspects of the disclosed embodiment the biometric sensor comprises one or more of a vision sensor, a heat-based sensor, an audio sensor, a motion sensor, and a biometric sensor.

In accordance with one or more aspects of the disclosed embodiment the biometric sensor comprises a wearable health device.

In accordance with one or more aspects of the disclosed embodiment the drive section provides more than one degree of freedom motion to the infant holder.

In accordance with one or more aspects of the disclosed embodiment an infant care apparatus comprises an infant holder; a drive section coupled to the infant holder, the drive section includes a motor configured to generate one or more of an action and a motion of the infant holder; a closed loop control system communicably coupled to the drive section, the closed loop control system comprising at least one biometric sensor configured to observe an active characteristic of an infant within the infant holder, and a controller configured to employ a neural network or state machine that is communicably coupled to the at least one biometric sensor and the drive section, the controller being configured to register a change in the active characteristic of the infant sensed by the at least one biometric sensor and generate, through the neural network or state machine, a responsive change in the one or more of an action and a motion of the infant holder based on the change in the active characteristic of the infant.

In accordance with one or more aspects of the disclosed embodiment the controller is configured to generate indicia to identify the change in one or more of the action characteristic of the infant and the motion characteristic of the infant sensed by the at least one biometric sensor.

It should be understood that the foregoing description is only illustrative of the aspects of the disclosed embodiment. Various alternatives and modifications can be devised by those skilled in the art without departing from the aspects of the disclosed embodiment. Accordingly, the aspects of the disclosed embodiment are intended to embrace all such alternatives, modifications and variances that fall within the scope of any claims appended hereto. Further, the mere fact that different features are recited in mutually different dependent or independent claims does not indicate that a combination of these features cannot be advantageously used, such a combination remaining within the scope of the aspects of the disclosed embodiment.

What is claimed is:

1. An infant care apparatus comprising:
an infant holder;
a drive section coupled to the infant holder and having a motor configured to generate one or more of an action and a motion of the infant holder;
a biometric sensor configured to observe at least one characteristic of an infant within the infant holder; and
a controller configured to employ a neural network or state machine that is communicably coupled to the biometric sensor and the drive section, where the controller registers sensor data from the biometric sensor and effects, through the neural network or the state machine, a change in the one or more of the action and the motion of the infant holder.

2. The infant care apparatus of claim 1, wherein the biometric sensor is configured to generate a sensor signal embodying the at least one characteristic of the infant, where the at least one characteristic is one or more of an action characteristic of the infant and a motion characteristic of the infant.

3. The infant care apparatus of claim 1, wherein the controller is configured to send a command to the motor based on the sensor data of the at least one characteristic sensed by the sensor.

4. The infant care apparatus of claim 1, wherein the controller generates a responsive change in the one or more of the action and the motion of the infant holder based on a change in one or more of an action characteristic of the infant and a motion characteristic of the infant sensed by the biometric sensor.

5. The infant care apparatus of claim 4, wherein the controller is configured to generate indicia to identify the responsive change in the one or more of the action and the motion of the infant holder.

6. The infant care apparatus of claim 5, wherein the indicia is one or more of aural indicia and visual indicia.

7. The infant care apparatus of claim 4, wherein the controller is configured to generate indicia to identify the change in one or more of the action characteristic of the infant and the motion characteristic of the infant sensed by the biometric sensor.

8. The infant care apparatus of claim 7, wherein the indicia is one or more of aural indicia and visual indicia.

9. The infant care apparatus of claim 1, wherein the biometric sensor comprises one or more of a vision sensor, a heat-based sensor, an audio sensor, a motion sensor, and a biometric sensor.

10. The infant care apparatus of claim 1, wherein the biometric sensor comprises a wearable health device.

11. The infant care apparatus of claim 1, wherein the drive section is configured to provide more than one degree of freedom motion to the infant holder.

12. A method for infant care with an infant care apparatus, the method comprising:
providing an infant holder;
providing a drive section coupled to the infant holder and having a motor configured to generate one or more of an action and a motion of the infant holder;
observing, with a biometric sensor, at least one characteristic of an infant within the infant holder; and
registering, with a controller configured for employing a neural network or state machine, sensor data from the sensor and effecting, with the neural network or state machine, a change in the one or more of the action and the motion of the infant holder.

13. The method of claim 12, wherein the biometric sensor generates a sensor signal embodying the at least one characteristic of the infant, where the at least one characteristic is one or more of an action characteristic of the infant and a motion characteristic of the infant.

14. The method of claim 12, wherein the controller sends a command to the motor based on the sensor data of the at least one characteristic sensed by the biometric sensor.

15. The method of claim 12, wherein the controller generates a responsive change in the one or more of the action and the motion of the infant holder based on a change in one or more of an action characteristic of the infant and a motion characteristic of the infant sensed by the biometric sensor.

16. The method of claim 15, wherein the controller generates indicia to identify the responsive change in the one or more of the action and the motion of the infant holder.

17. The method of claim 15, wherein the controller is configured to generate indicia to identify the change in one or more of the action characteristic of the infant and the motion characteristic of the infant sensed by the biometric sensor.

18. The method of claim 12, wherein the biometric sensor comprises one or more of a vision sensor, a heat-based sensor, an audio sensor, a motion sensor, and a biometric sensor.

19. The method of claim 12, wherein the biometric sensor comprises a wearable health device.

20. The method of claim 12, wherein the drive section provides more than one degree of freedom motion to the infant holder.

21. An infant care apparatus comprising:
an infant holder;
a drive section coupled to the infant holder, the drive section includes a motor configured to generate one or more of an action and a motion of the infant holder;
a closed loop control system communicably coupled to the drive section, the closed loop control system comprising:
at least one biometric sensor configured to observe an active characteristic of an infant within the infant holder, and
a controller configured to employ a neural network or state machine that is communicably coupled to the at least one biometric sensor and the drive section, the controller being configured to register a change in the active characteristic of the infant sensed by the at least one biometric sensor and generate, through the neural network or state machine, a responsive change in the one or more of an action and a motion of the infant holder based on the change in the active characteristic of the infant.

22. The infant care apparatus of claim 21, wherein the controller is configured to generate indicia to identify the change in one or more of the action characteristic of the infant and the motion characteristic of the infant sensed by the at least one biometric sensor.
